(19) United States
(12) Patent Application Publication
(10) Pub. No.: US 2020/0291369 A1
(43) Pub. Date: Sep. 17, 2020

(54) Improved CRISPR-Cas9 Genome Editing Tool

(71) Applicants: Erasmus University Medical Center Rotterdam; Wageningen Universiteit

(72) Inventors: Rogier Petrus Leonardus LOUWEN, Rotterdam (NL); John VAN DER OOST, Renkum (NL); Peter VAN BAARLEN, Wageningen (NL)

(21) Appl. No.: 16/084,014

(22) PCT Filed: Mar. 10, 2017
(86) PCT No.: PCT/NL2017/050154

§ 371 Date: Sep. 11, 2018

(30) Foreign Application Priority Data
Mar. 11, 2016 (NL) PCT/NL2016/050178

(51) Int. Cl. C12N 9/22; C12N 15/11; C12N 15/88; C12N 15/85

(52) U.S. Cl. CPC C12N 9/22; C12N 15/11; C12N 15/88; C12N 2310/3513; C12N 2800/80; C12N 2310/20; C12N 15/85

(57) ABSTRACT

The invention relates to a Cas-based, preferably Cas9-based nuclease complex, wherein the guide RNA sequence is irreversibly crosslinked to the Cas9 protein. The cross-link may be a covalent binding or a non-covalent binding. Such a complex may be used in delivering constructs to a cell that are capable of gene-editing. Use of this cross-linked complex will result in less off-targeting.

The invention relates to the field of genetics, more particular to the field of gene editing, especially gene editing through the CRISPR-Cas9 system.

CRISPR sequences are Clustered Regularly Interspaced Short Palindromic Repeat sequences that are present in bacteria and archaea. Initially these kind of sequences have been indicated as Short Regularly Spaced Repeats (SRSRs) (Mojica, F. J. et al., 2000, Mol. Microbiol. 36:244-246), but they have been renamed in the acronym CRISPR by Jansen et al. (Jansen, R. et al., 2002, Mol. Microbiol. 43:1565-1575). The function of the Class-2/Type II system ((CRISPR-associated protein 9; Cas9) has been revealed later by Barrangou, Horvath and Moineau, (Science, 315:1709-1712, 2007; Nature, 468:67-71, 2010), who showed that CRISPR-derived guides (crRNA) are used by CRISPR associated (Cas) proteins to provide immunity against viral infections. Subsequently, the group of Charpentier (Deltcheva, E. et al., Nature, 471:602-607, 2011) discovered that a second RNA (tracrRNA) forms a dual guide with the crRNA, that is essential for Cas9 functionality, i.e. cleavage of a complementary DNA sequence.

Later, Doudna and Charpentier, and Siksnys (Jinek, M. et al., 2012, Science 337:816-821; Gasjunas et al, 2012, Proc. Natl. Acad. Sci. 109:E2579-2586, 2012) showed that Cas9 can be used for genetic editing. Since then the CRISPR-Cas system has been studied extensively and currently it is one of the most promising tools in genetic engineering because of its ease of use (reviewed by Pennisi, E., 2013, Science 341:833-836, Young, S. 2014, MIT Technol. Review: http://www.technologyreview.com/review/524451/genome-surgery/; Mali, P. et al., 2013, Nature Meth. 10:957-963).

Cas9 (CRISPR associated protein 9) is an RNA-guided DNA endonuclease enzyme. Cas9 has gained traction in recent years because it can cleave nearly any sequence complementary to the guide RNA. The target specificity of Cas9 stems from the guide RNA:DNA complementarity and not modifications to the protein itself (like TALENs and Zinc-fingers), engineering Cas9 to target non-self DNA is straightforward. Versions of Cas9 that bind, but do not cleave cognate DNA, can be used to localize transcriptional activator or repressors to specific DNA sequences in order to control transcriptional activation and repression. While native Cas9 requires a guide RNA composed of two disparate RNAs that associate to make the guide—the CRISPR RNA (crRNA), and the trans-activating RNA (tracrRNA), Cas9 targeting has been simplified through the engineering of a chimeric single guide RNA. Scientists have suggested that Cas9-based gene drives may be capable of editing the genomes of entire populations of organisms. In 2015, scientists used Cas9 to modify the genome of human embryos for the first time.

One disadvantage of the CRISPR-Cas9 system is that in many cases off-targeting mutagenesis occurs, which can be described as introduction of double-strand breaks (DSBs) in DNA sequences that are not targeted by the guide RNA during gene-editing. This off-targeting is thought to be caused by non-specific interaction between the guide RNA and the target DNA, and/or by malfunctioning of the Cas9 enzyme. However, recently solutions have been provided after protein engineering of Cas9 (Slaymaker, I. et al., Science 351:84-88, 2016).

### SUMMARY OF THE INVENTION

The present inventors now found that the problem of off targeting is also dependent on Cas9 alone, but may be solved through the use of a Cas-based nuclease complex, wherein the guide RNA sequence is irreversibly crosslinked to the Cas protein. Preferably, in such a complex the guide RNA sequence comprises a CRISPR nucleic acid sequence. Further preferred is where the Cas protein is Cas9. Also preferred is a complex wherein the guide RNA is not derived from the same organism as the Cas protein.

In a preferred embodiment the Cas protein is derived from Pasteurella multocida, Streptococcus thermophilus, Streptococcus agalactiae, Streptococcus anginosus, Streptococcus bouis, Streptococcus canis, Streptococcus constellatus, Streptococcus dysgalactiae, Streptococcus equi, Streptococcus equinus, Streptococcus gallolyticus, Streptococcus infantarius, Streptococcus iniae, Streptococcus macacae, Streptococcus mitis, Streptococcus oxalis, Streptococcus gordonii, Streptococcus infantarius, Streptococcus macedonicus, Streptococcus parasanguinis, Streptococcus pasteurianus, Streptococcus pseudoporcinus, Streptococcus ratti, Streptococcus salivarius, Streptococcus sanguinis, Streptococcus suis, Streptococcus pyogenes, Streptococcus mutans, Streptococcus vestibularis, Pediococcus acidilactici, Staphylococcus aureaus, Staphylococcus lugdunensis, Staphylococcus pseudintermedius, Staphylococcus simulans, Escherichia coli, Neisseria bacilliformis, Neisseria cinerea, Neisseria flauescens, Neisseria lactamica, Neisseria meningitides, Neisseria wadsworthii, Listeria innocua, Francisella nouicida, Campylobacter jejuni, Campylobacter coli, Campylobacter lari, Helicobacter canadensis, Helicobacter cinaedi, Lactobacillus animalis, Lactobacillus farciminis, Lactobacillus buchneri, Lactobacillus casei, Lactobacillus coryniformis, Lactobacilus farciminis, Lactobacillus fermentum, Lactobacillus forum, Lactobacillus gasseri, Lactobacillus hominis, Lactobacillus iners, Lactobacillus jensenii, Lactobacillus johnsonii, Lactobacillus mucosae, Lactobacillus paracasei, Lactobacillus pentosus, Lactobacillus plantarum, Lactobacillus rhamnosus, Lactobacillus ruminis, Lactobacillus salivarius, Lactobacillus sanfranciscensis, Lactobaci/lus uersmoldensis, Legionella pneumophila, Listeria monocytogenes, Acidaminococcus intestine, Acidothermus cellulolyticus, Acidouorax ebreus, Actinobacillus minor, Actinobacillus pleuropneumoniae, Actinobacillus succinogenes, Actinobacillus suis, Actinomyces coleocanis, Actinomyces georgiae, Actinomyces naeslundii, Actinomyces turicensis, Acidouorax auenae, Akkermansia muciniphila, Alicycliphilus denitrificans, Alicyclobacillus hesperidum, Aminomonas pauciuorans, Anaerococcus tetradius, Anaerophaga thermohalophila, Bacillus cereus, Bacillus smithii, Bacillus thuringiensis, Bacteroides coprophilus, Bacteroides coprosuis, Bacteroides dorei, Bacteroides eggerthii, Bacteroides faecis, Bacteroides fluxus, Bacteroides fragilis, Bacteroides nordii, Bacteroides uniformis, Bacteroides uulgatus, Barnesiella intestinihominis, Bergeyella zoohelcum, Bifidobacterium bifidum, Bifidobacterium dentium, Bifidobacterium longum, Breuibacillus laterosporus, Caenispirillum salinarum, Capnocytophaga gingivalis, Capnocytophaga canimorsus, Capnocytophaga sputigena, Catellicoccus marimammalium, Catenibacterium mitsuokai, Clostridium perfringens, Clostridium spiroforme, Coprococcus catus, Coriobacterium glomerans, Corynebacterium accolens, Corynebacterium diphtheria, Dinoroseobacter shibae, Dorea longicatena, Dolosigranulum pigrum, Elusimicrobium minutum, Enterococcus faecalis, Enterococcus faecium, Enterococcus hirae, Enterococcus itaficus, Eubacterium dolichum, Eubacterium rectale, Eubacterium uentriosum, Eubacterium yurii, Facklamia hominis, Fibrobacter succinogenes, Filifactor alocis, Finegoldia magna, Flavobacterium branchiophilum, Flavobacterium columnare, Flavobacterium psychrophilum, Fluuiicola taffensis, Francisella tgularensis, Fructobacillus fructosus, Fusobacterium nucleaturn, Gardnerella vaginalis, Gemella haemolysans, Gemella morbillorum, Gluconacetobacter diazotrophicus, Gordonibacter pamelaeae, Haemophilus parainfluenzae, Haemophilus sputorum Helcococcus kunzii, Helicobacter mustelae, lndibacter alkaliphilus, lgnauibacterium album, llyobacter polytropus, Joostella marina, Kordia algicides, Leuconostoc gelidum, Methylosinus trichosporium, Mucilaginibacter paludis, Myroides injenensis, Myroides odoratus, Mobiluncus curtisii, Mobiluncus mulieris, Mycoplasma canis, Mycoplasma gallisepticium, Mycoplasma mobile, Mycoplasma ouipneumoniae, Mycoplasma synouiae, Niabella soli, Nitratifractor salsuginis, Nitrobacter hamburgensis, Odoribacter laneus, Oenococcus kitaharae, Ornithobacterium rhinotracheale, Parabacteroides johnsonii, Parasutterella excrementihominis, Paruibaculum lauamentiuorans, Phascolarctobacterium succinatutens, Planococcus antarcticus, Prevotella biuia, Prevotella buccae, Prevotella buccalis, Prevotella denticola, Prevotella histicola, Prevotella intermedia, Prevotella micans, Prevotella oralis, Prevotella nigrescens, Prevotella ruminicola, Prevotella stercorea, Prevotella tannerae, Prevotella timonensis, Prevotella ueroralis, Ralstonia syzygii, Rhodopseudomonas palustris, Rhodospirillum rubrum, Riemerella anatipestifer, Roseburia intestinalis, Ruminococcus albis, Ruminococcus lactaris, Scardouia inopinata, Scardouia wiggsiae, Solobacterium moorei, Sphaerochaeta_globus, Sphingobacterium spiritiuorum, Streptobacillus moniliformis, Sutterella wadsworthensis, Treponema denticola, Tistrella mobilis, Veillonella atypica, Veillonella paruula, Weeksella uirosa, Wolinella succinogenes or Zunongwangia profunda, more preferably from S. pyogenes, S. thermophilus, S. mutans, C. jejuni, F. nouicida, P. multocida or N. meningitides.

Further part of the invention is a complex as described above wherein the guide RNA is coupled to the Cas enzyme through an RNA linker molecule.

In a further preferred embodiment the guide RNA is covalently coupled to the Cas protein. Preferably the covalent coupling is established by UV irradiation. It is also preferred when the coupling is made via the backbone of the RNA molecule.

In an alternative embodiment the guide RNA is non-covalently complexed with the Cas protein.

Further part of the invention is a method for delivering a construct capable of gene editing to a eukaryotic cell, comprising the steps of:

a. providing a complex as described above; and

b. introducing said eukaryotic cell with said vector.

Also part of the invention is a method for gene editing a eukaryotic cell comprising providing a complex as described above to said cell. Preferably in these methods said cell is part of an organism, preferably wherein the organism is selected from the group of fungi, algae, plants and animals, including human. Also part of the invention is the use of a cross-linked complex of Cas9 and a guide RNA for gene-editing, preferably gene-editing of eukaryotic cells.

It has further been shown that the problem of off-targeting and toxicity from (unbound) Cas9 appearance in the cell may also be solved by providing an already complexed Cas9-gRNA system to the cell. Such a system can preferably be transferred to the cell via lipofection or other transfection methods. A further preferred embodiment for at least partially solving the problem of the prior art is to overexpress the gRNA in such a way

### LEGENDS TO THE FIGURES

FIG. 1A shows Caco-2 cells grown and differentiated for 19 days on a Transwell filter and stained with Haematoxylin and Eosin. The Caco-2 cells were judged for damage using a light microscope and a 40× magnification. The samples on the left (NCTC11168, GB2, GB11 and GB19) are Caco-2 cells infected with wild type strains (Cas+) and on the right with their corresponding isogenic Δcas9 mutants (Cas9−). Presence of Cas9 was accompanied with cell swelling and damage, whereas this was absent when Cas9 was absent, than the cells looked identical to the uninfected Caco-2 cells as visualized in the control picture.

FIG. 1B shows HELA cells infected overnight with wild type strains NCTC11168 (green), GB2 (Dark red), GB11 (orange) and GB19 (Blue) and their corresponding isogenic Δcas9 mutants. HELA cells were stained with neutral red after which the OD was measured at 540nm. The neutral red assay established that HELA cells infected with wild type strains (Cas9+) were less viable compared to HELA cells infected with their isogenic Δcas9 mutants (Cas9−) as revealed by an increased OD540nm signal. Microscopic pictures at a 40× magnification of HELA cells infected with GB11, GB19 wild type (Cas9+) strains and their Δcas9 mutants (Cas9−) established that in the presence of Cas9 cells lost their viability.

FIG. 1C. U2OS, HELA, Caco-2 and K562 cells were grown to confluence in a 12-well plate and infected with the wild type strain GB11 (Cas9+), its isogenic GB11Δcas9 mutant (Cas9−) or its complemented GB11Δcas9Δ mutant variant (Cas9+). When Cas9 was present U2OS, HELA, Caco-2 and K562 cells became apoptotic/necrotic, but the isogenic Δcas9 mutants lost the ability to kill the U2OS, HELA, Caco-2 and K562 cells.

FIG. 1D K562 cell death was further quantified by FACS analyses of which the values were put into an XY graph, revealing that GB11 and GB11Δcas9Δ infected K562 cells resulted in significant more death cells (** p<0.0001) then when infected with GB11Δcas9 or left untreated. Experiment was repeated 5 times and at each time point (shown in hours) the mean and the standard error of the mean is shown of the measured percentage of apoptotic cells.

FIG. 2 visualizes the activation of the DNA damage pathway (P53) in Caco-2 cells upon infection with a wild type Campylobacter jejuni strain that harbors Cas9. This activation was found to be absent when the isogenic Δcas9 mutant of this strain was allowed to infect Caco-2 cells. Indicating that Cas9 is required to induce DNA damage and apoptosis.

FIG. 3 shows the 11168 and GB11 strain with or without the fusion of mCherry to Cas9. Both variants were allowed to infect the U2OS cells overnight. U2OS cells infected with the variants harboring mCherry fused to Cas9 were found to become redish in the cytoplasm and nucleus/nucleolus. In cells with a red nucleus/nucleolus this organelle was found to be severely damaged as found with the DAPI staining (see white arrows)

FIG. 4 shows Caco-2 cells (GFP-CjCas9) and U2OS cells (GFP-FnCas9) that were transfected with a plasmid that forced the expression of Campylobacter jejuni Cas9 or Francisella nouicida Cas9 fused to GFP. White arrow shows that both Cas9 proteins are able to localize into the eukaryotic nucleus.

FIG. 5A shows a Westernblot from the cytoplasmic (CP) or nuclear fraction (NP) obtained from HEK293T cells. The HEK293T cells were transfected with the pEGFP-C1 vector that harbored Cas9 from Campylobacter jejuni, Streptococcus pyogenes, Neisseria meningitidis and Francisella nouicida. As a control pEGFP-C1 transfected and non-transfected HEK293T cells were used. GFP expression was detected with an anti-GFP antibody between 25-35kDA. With this same antibody between 135-150kDa the GFP-Cas9 fusion protein of the above mentioned bacterial species was detected in both the cytoplasmic and nuclear protein fractions, showing that all bacterial Cas9 proteins are able to localize into the eukaryotic nucleus.

FIG. 5B GAPDH is detected with an anti-GAPDH antibody establishing that the separation of the nuclear fraction from the cytoplasmic fraction as visualized for Cas9 in (FIG. 5A) was sufficient. This figure thus establishes that leakage of cytoplasmic proteins as visualized by GAPDH to the nuclear fraction in which GAPDH is almost absent was reduced to a minimum. This control thus confirms that Cas9 of different bacterial species efficiently localize into the nucleus.

FIG. 6A U2OS cells were infected with wild type Campylobacter jejuni strains (11168 or GB11), their Δcas9 mutants and their complemented Δcas9Δ mutants, radiated with 1 Gy or left untreated. After six hours cells were fixated with 4% paraformaldehyde and stained for y-H2AX to detect the induction of double stranded breaks. For cells radiated with 1 Gy the fixation occurred 30 minutes after radiation. U2OS cells infected with Campylobacter jejuni strains harboring Cas9 (wild type or complemented Δcas9Δ mutants) showed a significant increase in y-H2AX staining as compared to the U2OS cells infected with the Δcas9 mutants or untreated cells. U2OS cells radiated with 1 Gy also showed an increase in y-H2AX foci, but to a lesser extend as seen for U2OS cells infected with Cas9 positive Campylobacter jejuni strains.

FIG. 6B U2OS cells were transfected with the pEGFP-C1 plasmid containing Cas9 of Francisella nouicida, Neisseria meningitidis or Streptococcus pyogenes. After 24 hours cells were fixated with 4% paraformaldehyde and stained for y-H2AX. Nucleus was stained with DAPI, Cas9 expression is visualized by GFP. All three Cas9 proteins of Francisella nouicida, Neisseria meningitidis or Streptococcus pyogenes were able to induce double stranded breaks in the nuclei as visualized by the y-H2AX staining without any addition of a guide RNA.

FIG. 7 To visualize the nuclear localization of Campylobacter jejuni Cas9 and its ability to induce double stranded breaks, U2OS cells were transfected with pEGFP-C1, pEGFP-C1 fused to the NLS of Campylobacter jejuni Cas9, pEGFP-C1 fused to an inactive variant of Campylobacter jejuni Cas9 (dCas9) or pEGFP-C1 fused to an active Campylobacter jejuni Cas9. pEGFP-C1 alone (GFP-CT) revealed expression of GFP mainly in the cytoplasm of the eukaryotic cell with some leakage to the nucleus. pEGFP-C1 fused to the NLS of Campylobacter jejuni Cas9 (GFP-NLS) demonstrated that GFP localised specifically in the nucleus and nucleolus of the U2OS cells. pEGFP-C1 fused to dCas9 of Campylobacter jejuni (GFP-dCas9) showed the localisation of dCas9 to the cytoplasm and nucleus of the U2OS cell. pEGFP-C1 fused to Cas9 of Campylobacter jejuni (GFP-Cas9) established that Cas9 localises to the nucleus and nucleolus of the U2OS cells. The y-H2AX staining established that an active Cas9 is required to induce double stranded DNA breaks as visualized by the y-H2AX staining and is able to do so without any addition of a guide RNA. Visualization occurred after 48 hours at a 100× magnification, white arrow shows an example of a nuclear localisation related Campylobacter jejuni Cas9 dependent phenotype.

FIG. 8 U2OSmCherryRAD52 positive cells were grown to confluence on a 6 well plate. U2OS cells were infected with the Campylobacter jejuni strain GB11, GB11Δcas9 mutant or GB11Δcas9Δ, complemented mutant at a MOI of 100. After 48 hours the U2OS cells were judged at a 40× magnification. GB11 and its GB11Δcas9Δ, complemented mutant both harboring Cas9 were found to induce significant cell damage/death. U2OS cells infected with the GB11Δcas9 mutant looked unaffected and showed mCherryRAD52 spots in the nucleus an indication for active DNA repair.

FIG. 9A-D shows pictures of Campylobacter jejuni Cas9 and PAM motif dependent double stranded breaks extracted from U2OS cells using the BLESS technique. FIG. 9A shows a double stranded break induced in the intron of gene ZZZ3 in a PAM motif dependent manner as visualized in red at the 3′ site of the break. The break induced by the wild type strain GB11 could be complemented after infection of U2OS cells using the GB11Δcas9Δ complemented mutant. FIG. 9B shows a double stranded break induced by GB11 in a non-coding area in a PAM motif specific manner as visualized in red at the 3′ site of the break and could be complemented by the GB11Δcas9Δ, complemented mutant in U2OS cells. FIG. 9C shows a double stranded break induced by GB11 in the intron of ST3GAL3 in an area that also harbors a small non-coding RNA (HSA-MIR 6079). This break could be complemented by the GB11Δcas9Δ, complemented mutant in U2OS cells. FIG. 9D shows a double stranded break induced by GB11 in the intron of GNG12-AS1 in an area that also harbors a small non-coding RNA (SSU-tRNA_Hsa) transcribed from a human repeat and is reverse complementary to the transcription of the mRNA of GNG12-AS1. This break could be complemented by the GB11Δcas9Δ complemented mutant in U2OS cells. The examples of Campylobacter jejuni Cas9 dependent breaks were absent in the GB11Δcas9 mutant infected U2OS cells and uninfected U2OS cells. The breaks were induced after 6 hours of infection. Additional examples of infection related and C. jejuni Cas9 dependent double stranded DNA breaks can be found in Table 1.

FIG. 9E and 9F show pictures of Campylobacter jejuni Cas9 dependent breaks after plasmid transfection. Plasmid pEGFP-C1 fused to C. jejuni Cas9 or an inactive form of Cas9 or plasmid pCDNA3.1 containing the restriction enzyme I-SceI with a NLS signal were transfected to U2OS cells, or additionally U2OS cells were radiated with 1 Gy or left untreated. Double stranded DNA breaks present in U2OS cells were isolated using the BLESS protocol after 30 minutes of radiation exposure (PC_1Gy_25012016); 24 hours for GFPCas9_250102016; GFPdCas9_25012016; PC_I-Scel_25012016 and NC_210102016 or 48 hours for GFPCas9(48h)_25012016 after plasmid transfection. In FIG. 9E and 9F a C. jejuni Cas9 PAM motif dependent (visualized in red) double stranded break is identified in the intron of LUZP1 after 24 hours and in the intron of ASAP3 harboring at the break site a reverse complementary human repeat after 48 hours. These breaks were found to be absent in any other sample. Some of the breaks induced after plasmid transfection by Campylobacter jejuni Cas9 in U2OS cells could be confirmed in the samples harboring Campylobacter jejuni dependent double stranded DNA breaks obtained after infection of U2OS cells. These plasmid based Cas9 complemented double stranded DNA breaks can be found in Table 2.

FIG. 9G and 9H show pictures of Streptococcus pyogenes Cas9 and PAM motif dependent (visualized in red) double stranded DNA breaks after plasmid transfection. pCDNA3.1 harboring Cas9 of S. pyogenes Cas9 was transfected to U2OS cells. Double stranded breaks were obtained using BLESS after 24 and 48 hours and were absent in the samples containing breaks from (PC_1Gy_25012016); PC_I-Scel_25012016 and NC_210102016. FIG. 9G shows a double stranded DNA break induced by Streptococcus pyogenes Cas9 after 24 hours in the intron of gene UBE4B in an area that harbors a reverse complementary repeat named AluSz. FIG. 9H shows a double stranded DNA break induced by Streptococcus pyogenes Cas9 after 24 and 48 hours in the intron CTNNBIP1 in which the break at 24 hours is PAM motif dependent (visualized in red), but at 48 hours (likely) PAM motif independent.

FIG. 10 shows FACS data with on the x-axis FL-1a normal (Q2-LL) or GFP positive cells (Q2-LR) and on the y-axis FL-3a normal (Q2-LL), dead cells (Q2-UL) or GFP positive dead cells (Q2-UR) 72 hours post-transfections. Cas9(NLS) is the human optimized SpyCas9 commonly used for genome editing purposes. Cas9(Bac) is the bacterial SpyCas9 from which the human optimized SpyCas9 is derived. The FACS data reveals that also the bacterial SpyCas9 without an additional added nuclear localisation signal can actively edit eukaryotic cells.

FIG. 11 shows at a 20× magnification in bright field (top) and GFP fluorescent (bottom) pictures of control cells (Plain), transfected with gRNA only, donor GFP template only, SpyCas9 only, and cells transfected with all three components. Only in the presence of all three components GFP expression is observed.

FIG. 12 depicts the percentage of apoptotic cells (y-axis) when cells are transfected with Cas9 proteins in the presence or absence of guide RNA. The white bars are untransfected cells or exposed to the transfection agent (lipofectamine 2000) only; black bars are eukaryotic cells exposed to the transfection agent in combination with different concentrations of the bacterial SpyCas9(Bac) protein and in the presence or absence of the guide RNA; grey bars are eukaryotic cells exposed to the transfection agent in combination with different concentrations of the human SpyCas9(NLS) protein and in the presence or absence of the guide RNA.

FIG. 13 Western blot detection showing the amount of CjCas9 protein in the nuclear (NP) and cytoplasmic (CP) protein fractions of eukaryotic cells that were obtained 24 hours after transfection with a plasmid expressing the CjCas9 or a guide RNA that targets GFP and did thus not possess a direct target in the eukaryotic genome. Anti-GAPDH was used to visualize the quality of the NP and CP separation and equal loading. C. jejuni anti-Cas9 polyclonal antibody (anti-CjCas9) and anti-y-H2AX were used to detect the Cas9 accumulation and DNA damage induction in nucleus.

FIG. 14 shows an electrophoresis gel loaded with the gRNA alone (dark grey arrow); a combination of the gRNA with the bacterial SpyCas9(Bac) or human optimized SpyCas9(NLS) (white arrow); or a combination of the gRNA with the bacterial SpyCas9(Bac) or human optimized SpyCas9(NLS) after UV-crosslinking at different intensities (black arrow). The mobility gel shift assay shows that UV crosslinking of the gRNA and the bacterial or human optimized SpyCas9 affects its mobility.

FIG. 15 shows FACS data with on the x-axis FL-1a normal (Q2-LL) or GFP positive cells (Q2-LR) and on the y-axis FL-3a normal (Q2-LL), dead cells (Q2-UL) or GFP positive dead cells (Q2-UR) 72 hours post-transfections. Cas9(NLS) is the human optimized SpyCas9 commonly used for genome editing purposes. The dCas9(NLS) is the heat inactivated Cas9(NLS). The FACS data reveals that SpyCas9(NLS) can actively edit eukaryotic cells, as visualized by GFP expressing cells, after UV-crosslinking

### DETAILED DESCRIPTION

Definitions

All technical and scientific terms used herein have the same meaning as commonly understood by one of ordinary skill in the art to which this invention belongs, unless the technical or scientific term is defined differently herein.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

“Genomic DNA” refers to the DNA of a genome of an organism including, but not limited to, the DNA of the genome of a bacterium, fungus, archaea, plant or animal

“Manipulating” DNA encompasses binding, nicking one strand, or cleaving (i.e., cutting) both strands of the DNA, or encompasses modifying the DNA or a polypeptide associated with the DNA (e.g., amidation, methylation, etc.) . Manipulating DNA can silence, activate, or modulate (either increase or decrease) the expression of an RNA or polypeptide encoded by the DNA.

“Gene-editing” refers to the process of changing the genetic information present in the genome of a cell. This gene-editing may be performed by manipulating genomic DNA, resulting in a modification of the genetic information. Such gene-editing may or may not influence expression of the DNA that has been edited.

A “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. As is known in the art, a stem-loop structure does not require exact base-pairing. Thus, the stem may include one or more base mismatches. Alternatively, the base-pairing may be exact, i.e. not include any mismatches.

By “hybridizable” or “complementary” or “substantially complementary” it is meant that a nucleic acid (e.g. RNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (r), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 55%, at least 60%, at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining non-complementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide) refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10⁻⁸ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd. By “binding domain” it is meant a protein domain that is able to bind non-covalently to another molecule. A binding domain can bind to, for example, a DNA molecule (a DNA-binding protein), an RNA molecule (an RNA-binding protein) and/or a protein molecule (a protein-binding protein). In the case of a protein domain-binding protein, it can bind to itself (to form homodimers, homotrimers, etc.) and/or it can bind to one or more molecules of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine- isoleucine, phenylalanine-tyrosine, lysine-arginine, alanine-valine, and asparagine-glutamine

A polynucleotide or polypeptide is “homologous” to, or has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), available over the world wide web at sites including http://blast.ncbi.nlm nih.gov/Blast.cgi?CMD=Web&PAGE_TYPE=BlastDocs&DOC_TY PE=Download. See, e.g., Altschul et al. (1990), J. Mol. Biol. 215:403-10. Sequence alignments standard in the art are used according to the invention to determine amino acid residues in a Cas9 orthologue that “correspond to” amino acid residues in another Cas9 orthologue. The amino acid residues of Cas9 orthologs that correspond to amino acid residues of other Cas9 orthologs appear at the same position in alignments of the sequences.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence a transcription initiation site will be found, as well as protein binding domains responsible for the binding of RNA polymerase. Eukaryotic promoters will often, but not always, contain so-called “TATA” boxes and “CAT” boxes. Various promoters, including inducible promoters, may be used to drive the vectors as described in the present disclosure.

A promoter can be a constitutively active promoter (i.e., a promoter that is constitutively in an active (“ON”) state), it may be an inducible promoter (i.e., a promoter whose state, active (“ON”) or inactive (“OFF”), is controlled by an external stimulus, e.g., the presence of a particular temperature, compound, or protein). It may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hibernation in plants).

Where in the prior art suitable promoters for the expression of the Cas proteins have been derived from viruses (which can therefore be referred to as viral promoters) also preferred are the promoters that are used to express the Cas9 protein in wild-type bacteria. It is also possible that a promoter that is known to drive Cas9 expression in one bacterium is used to drive the expression of a Cas9 protein derived from a different (species) of bacterium. In such case, it is said that said promoter is heterologous with respect to the Cas9 protein. Alternatively, they can be derived from any organism, including prokaryotic or eukaryotic organisms. Suitable promoters can be used to drive expression by any RNA polymerase (e.g., pol I, pol II, pol III). Exemplary viral promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter. Human promoters comprise a human U6 small nuclear promoter (U6) (Miyagishi et al. , Nature Biotechnology 20, 497-500 (2002)), an enhanced U6 promoter (e.g., Xia et al., Nucleic Acids Res. 2003 Sep 1;31(17)), a human H1 promoter (H1), and the like.

Examples of inducible promoters include, but are not limited to T7 RNA polymerase promoter, T3 RNA polymerase promoter, isopropyl-beta-D-thiogalactopyranoside (IPTG)-regulated promoter, lactose induced promoter, heat shock promoter, tetracycline-regulated promoter, steroid-regulated promoter, metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc.

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSEN02, X51956); an aromatic amino acid decarboxylase (MDC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med.16(10): 1161-1166); a serotonin receptor promoter (see, e.g., GenBank S62283); a tyrosine hydroxylase promoter (TH) (see, e.g., Oh et al. (2009) Gene Ther 16:437; Sasaoka et al. (1992) Mol. Brain Res.16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase II-alpha (CamKIM) promoter (see, e.g., Mayford et al. (1996) Proc. Natl. Acad. Sci. USA 93: 13250; and Casanova et al. (2001) Genesis 31:37); a CMV enhancer/platelet-derived growth factor-p promoter (see, e.g., Liu et al. (2004) Gene Therapy 11:52-60); and the like.

Adipocyte-specific spatially restricted promoters include, but are not limited to aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25: 1476; and Sato et al. (2002) J. Biol. Chem. 277: 15703); a stearoyl

CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262: 187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to an SM22a promoter (see, e.g., Akyiirek et al. (2000) Mol. Med. 6:983; and U.S. Pat. No. 7,169,874); a smoothelin promoter (see, e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (see, e.g., Kim, et al. (1997) Mol. Cell. Biol. 17, 2266- 2278; Li, et al., (1996) J. Cell Biol. 132, 849-859; and Moessler, et al. (1996) Development 122, 2415- 2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9: 1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-protein coding sequence (e.g., guide RNA) or a protein coding sequence (e.g., Cas9 polypeptide) and/or regulate translation of an encoded polypeptide.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “chimeric” as used herein as applied to a nucleic acid or polypeptide refers to two components that are defined by structures derived from different sources. For example, where “chimeric” is used in the context of a chimeric polypeptide (e.g., a chimeric Cas9 protein), the chimeric polypeptide includes amino acid sequences that are derived from different polypeptides. A chimeric polypeptide may comprise either modified or naturally-occurring polypeptide sequences (e.g., a first amino acid sequence from a modified or unmodified Cas9 protein; and a second amino acid sequence other than the Cas9 protein). Similarly, “chimeric” in the context of a polynucleotide encoding a chimeric polypeptide includes nucleotide sequences derived from different coding regions (e.g., a first nucleotide sequence encoding a modified or unmodified Cas9 protein; and a second nucleotide sequence having another function, such as a nuclear localization signal).

The term “chimeric polypeptide” refers to a polypeptide which is not naturally occurring, e.g., is made by the artificial combination (i.e., “fusion”) of two otherwise separated segments of amino sequence through human intervention. A polypeptide that comprises a chimeric amino acid sequence is a chimeric polypeptide. Some chimeric polypeptides can be referred to as “fusion variants.”

“Heterologous,” as used herein, means a nucleotide or peptide that is not found in the native nucleic acid or protein, respectively. For example, in a chimeric Cas9 protein, the RNA-binding domain of a naturally-occurring bacterial Cas9 polypeptide (or a variant thereof) may be fused to a heterologous polypeptide sequence (i.e. a polypeptide sequence from a protein other than Cas9 or a polypeptide sequence from another organism). The heterologous polypeptide may exhibit an activity (e.g., enzymatic activity) that will also be exhibited by the chimeric Cas9 protein (e.g., nuclease activity, methyltransferase activity, acetyltransferase activity, kinase activity, etc.). A heterologous nucleic acid may be linked to a naturally-occurring nucleic acid (or a variant thereof) (e.g., by genetic engineering) to generate a chimeric polynucleotide encoding a chimeric polypeptide. As another example, in a fusion variant Cas9 site-directed polypeptide, a variant Cas9 site-directed polypeptide may be fused to a heterologous polypeptide (i.e. a polypeptide other than Cas9), which exhibits an activity that will also be exhibited by the fusion variant Cas9 site-directed polypeptide. A heterologous nucleic acid may be linked to a variant Cas9 site-directed polypeptide (e.g., by genetic engineering) to generate a polynucleotide encoding a fusion variant Cas9 site-directed polypeptide. “Heterologous,” as used herein, additionally means a nucleotide or polypeptide in a cell that is not its native cell.

The term “cognate” refers to two biomolecules that normally interact or co-exist in nature.

“Recombinant,” as used herein, means that a particular nucleic acid (DNA or RNA) or vector is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below).

Alternatively, DNA sequences encoding RNA (e.g., guide RNA) that is not translated may also be considered recombinant Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence.

Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

The term “recombinant bacteria” means bacteria which have been modified by a change in the total nucleic acid content which is contained in such bacteria. This change can be effected by introduction of a heterologous nucleic acid, but it may also be effected by a non-naturally induced change in the nucleic acid, such as a mutation, wherein this mutation can comprises a replacement of nucleic acids, an insertion of nucleic acids or a deletion of nucleic acids.

An “expression vector” is a replicon, such as plasmid, phage, virus, or cosmid, to which another DNA segment, i.e. an “insert”, may be attached so as to bring about the replication of the attached segment in a cell. A “vector” in the present application is an organism, such as viruses or bacteria that may be used to transfer nucleic acids, proteins and/or bacteria into another organism. Especially in the present invention a vector is used to transfer the crosslinked Cas9-DNA complex into the target cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. Alternatively, a cell has been “genetically modified” or “transformed” or “transfected” by introducing into said cell a crosslinked Cas9-DNA complex as defined in the present application.

In prokaryotes, yeast, and mammalian cells for example, the transforming DNA may be maintained on an episomal element such as a plasmid. With respect to eukaryotic cells, a stably transformed cell is one in which the transforming DNA or (a part of) the DNA part that is present on the Cas9-DNA complex has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include e.g., viral or bacteriophage infection, transfection, conjugation, protoplast fusion, lipofection, electroporation, calcium phosphate precipitation, polyethyleneimine (PE1)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep 13. pii: S0169-409X(12)00283-9. doi: 10.1016/j.addr.2012.09.023), and the like. Numerous transfection methods have been developed to transfer proteins and other macromolecules across the plasma membrane efficiently. These include physical methods, such as electroporation, sonoporation, cell squeezing, magnetofection, optical transfection, impalefection and microinjection, as well as a chemical or biological carrier-mediated methods. Chemical transfection reagents such as cationic lipids or polymers are widely used, either alone or in combination with scaffolds. Biological methods include delivery with cell-penetrating protein domain fusions such as trans-activator of transcription protein from human immunodeficiency virus, VP22 or Antennapedia peptides. Certain proteins such as zinc-finger nucleases, which are used for targeted genome modification, even appear to have intrinsic cell-penetrating properties. For the specific transfer of the Cas-DNA complexes of the present invention transfer systems that are known for proteins may be advantageously used. Such systems comprise penetrating peptides (Wagstaff et al., Curr Med Chem. 2006;13:1371-1387; Mae et al., Curr Opin Pharmacol. 2006;6:509-514 and e.g. HSV-VP22 as described by Xiong et al., BMC Neuroscience 2007, 8:50), some of which may be commercially available (e.g. the Xfect™ Protein Transfection Reagent from Clontech), proteoliposomes (Liguouri L., Meth Enzymol. 2009;465:209-223) and lipid based transfection systems (e.g. the Fuse-it-P™ system from Ibidi, Planegg, Germany; PULSin® from Source Bioscience, Nottingham, UK)) and viral based vesicle systems, such as from Vaccinia virus (Temchura et al., 2008 July 4;26(29-30):3662-72), capsids from polyoma virus (Bertling, W., Buiosci. Rep. 1987, 7(2):107-112) or by virus-derived nanovesicles VSV-G induced nanovesicles as described in Mangeot et al., Molecular Therapy (2011) 19 9, 1656-1666).

The choice of method of genetic modification is generally dependent on the type of cell being transformed and the circumstances under which the transformation or transfection is taking place (e.g., in vitro, ex vivo, or in vivo). A general discussion of these methods can be found in Ausubel, et al., Short Protocols in Molecular Biology, 3rd ed., Wiley & Sons, 1995.

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed or transfected by the nucleic acid or a complex with said nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a bacterial host cell is a genetically modified bacterial host cell by virtue of introduction into a suitable bacterial host cell of an exogenous nucleic acid or a complex with such a nucleic acid (e.g., a plasmid, vector or recombinant expression vector) and a eukaryotic host cell is a genetically modified eukaryotic host cell (e.g., a mammalian germ cell), by virtue of introduction into a suitable eukaryotic host cell of an exogenous nucleic acid or a complex with such a nucleic acid.

A “target DNA” as used herein is a DNA polynucleotide that comprises a “target site” or “target sequence.” The terms “target site.” “target sequence,” “target protospacer DNA. ” or “protospacer-like sequence” are used interchangeably herein to refer to a nucleic acid sequence present in a target DNA to which a DNA-targeting segment of a guide RNA will bind, provided sufficient conditions for binding exist. For example, the target site (or target sequence) 5′-GAGCATATC-3′ within a target DNA is targeted by (or is bound by, or hybridizes with, or is complementary to) the RNA sequence 5′-GAUAUGCUC-3′. Suitable DNA/RNA binding conditions include physiological conditions normally present in a cell. Other suitable DNA/RNA binding conditions (e.g., conditions in a cell-free system) are known in the art; see, e.g., Sambrook, supra. The strand of the target DNA that is complementary to and hybridizes with the guide RNA is referred to as the “complementary strand” and the strand of the target DNA that is complementary to the “complementary strand” (and is therefore not complementary to the guide RNA) is referred to as the “non-complementary strand.” By “site-directed modifying polypeptide” or “RNA-binding site-directed polypeptide” or “RNA-binding site-directed modifying polypeptide” or “site-directed polypeptide” it is meant a polypeptide that binds RNA and is targeted to a specific DNA sequence. A site-directed modifying polypeptide as described herein is targeted to a specific DNA sequence by the RNA molecule to which it is bound. The RNA molecule comprises a sequence that binds, hybridizes to, or is complementary to a target sequence within the target DNA, thus targeting the bound polypeptide to a specific location within the target DNA (the target sequence).

By “cleavage” is meant the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guide RNA and a site-directed modifying polypeptide is used for targeted double- stranded DNA cleavage.

“Nuclease” and “endonuclease” are used interchangeably herein to mean an enzyme which possesses endonucleolytic catalytic activity for DNA cleavage.

By “cleavage domain” or “active domain” or “nuclease domain” of a nuclease it is meant the polypeptide sequence or domain within the nuclease enzyme which possesses the catalytic activity for DNA cleavage. A cleavage domain can be contained in a single polypeptide chain or cleavage activity can result from the association of two (or more) polypeptides. A single nuclease domain may consist of more than one isolated stretch of amino acids within a given polypeptide.

The RNA molecule that binds to the site-directed modifying polypeptide and targets the polypeptide to a specific location within the target DNA is referred to herein as the “guide RNA” or “guide RNA polynucleotide” (also referred to herein as a “guide RNA” or “gRNA”). A guide RNA comprises two segments, a “DNA-targeting segment” and a “protein-binding segment.” By “segment” it is meant a segment/section/region of a molecule, e.g., a contiguous stretch of nucleotides in an RNA. A segment can also mean a region/section of a complex such that a segment may comprise regions of more than one molecule. For example, in some cases the protein-binding segment (described below) of a guide RNA is one RNA molecule and the protein-binding segment therefore comprises a region of that RNA molecule. In other cases, the protein-binding segment (described below) of a guide RNA comprises two separate molecules that are hybridized along a region of complementarity. As an illustrative, non-limiting example, a protein-binding segment of a guide RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a target DNA (the complementary strand of the target DNA) designated the “protospacer-like” sequence herein. The protein-binding segment (or “protein- binding sequence”) interacts with a site-directed modifying polypeptide. When the site-directed modifying polypeptide is a Cas9 or Cas9 related polypeptide (described in more detail below), site-specific cleavage of the target DNA occurs at locations determined by both (i) base-pairing complementarity between the guide RNA and the target DNA; and (ii) a short motif (referred to as the protospacer adjacent motif (PAM)) in the target DNA.

The protein-binding segment of a guide RNA comprises, in part, two complementary stretches of nucleotides that hybridize to one another to form a double stranded RNA duplex (dsRNA duplex).

In some embodiments, a nucleic acid (e.g., a guide RNA, a nucleic acid comprising a nucleotide sequence encoding a guide RNA; a nucleic acid encoding a site-directed polypeptide; etc.) comprises a modification or sequence that provides for an additional desirable feature (e.g., modified or regulated stability; subcellular targeting; tracking, e.g., a fluorescent label; a binding site for a protein or protein complex; etc.). Non-limiting examples include: a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like), more specifically a modification or sequence that provides a binding site for cross-linking it to a nuclease enzyme; and combinations thereof.

In some embodiments, a guide RNA comprises an additional segment at either the 5′ or 3′ end that provides for any of the features described above. For example, a suitable third segment can comprise a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and protein complexes); a stability control sequence; a sequence that forms a dsRNA duplex (i.e., a hairpin)); a sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) more specifically a modification or sequence that provides a binding site for cross-linking it to a nuclease enzyme; and combinations thereof.

A guide RNA and a site-directed modifying polypeptide (i.e., site-directed polypeptide) form a complex (i.e., bind via non-covalent interactions). The guide RNA provides target specificity to the complex by comprising a nucleotide sequence that is complementary to a sequence of a target DNA. The site-directed modifying polypeptide of the complex provides the site-specific activity. In other words, the site-directed modifying polypeptide is guided to a target DNA sequence (e.g. a target sequence in a chromosomal nucleic acid; a target sequence in an extrachromosomal nucleic acid, e.g. an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment of the guide RNA.

In most embodiments, a guide RNA comprises two separate RNA molecules (RNA polynucleotides: an “activator-RNA” and a “targeter-RNA”, see below) and is referred to herein as a “double-molecule guide RNA” or a “two-molecule guide RNA.” In other embodiments, the guide RNA is a single RNA molecule (single RNA polynucleotide) and is referred to herein as a “single-molecule guide RNA,” a “single-guide RNA,” or an “sgRNA.” The term “guide RNA” or “gRNA” is inclusive, referring both to double-molecule guide RNAs and to single-molecule guide RNAs (i.e., sgRNAs).

A two-molecule guide RNA comprises two separate RNA molecules (a “targeter-RNA” and an “activator-RNA”). Each of the two RNA molecules of a two-molecule guide RNA comprises a stretch of nucleotides that are complementary to one another such that the complementary nucleotides of the two RNA molecules hybridize to form the double stranded RNA duplex of the protein-binding segment.

An exemplary two-molecule guide RNA comprises a crRNA-like (“CRISPR RNA” or “targeter- RNA”) molecule (which includes a CRISPR repeat or CRISPR repeat-like sequence) and a corresponding tracrRNA-like (“trans-activating CRISPR RNA” or “activator-RNA” or “tracrRNA”) molecule. A crRNA-like molecule (targeter-RNA) comprises both the DNA-targeting segment (single stranded) of the guide RNA and a stretch (“duplex-forming segment”) of nucleotides that forms one half of the dsRNA duplex of the protein-binding segment of the guide RNA. A corresponding tracrRNA-like molecule (activator-RNA) comprises a stretch of nucleotides (duplex-forming segment) that forms the other half of the dsRNA duplex of the protein-binding segment of the guide RNA. In other words, a stretch of nucleotides of a crRNA-like molecule are complementary to and hybridize with a stretch of nucleotides of a tracrRNA-like molecule to form the dsRNA duplex of the protein-binding domain of the guide RNA. As such, each crRNA-like molecule can be said to have a corresponding tracrRNA-like molecule. The crRNA-like molecule additionally provides the single stranded DNA-targeting segment. Thus, a crRNA-like and a tracrRNA-like molecule (as a corresponding pair) hybridize to form a guide RNA. A double-molecule guide RNA can comprise any corresponding crRNA and tracrRNA pair.

A single-molecule guide RNA comprises two stretches of nucleotides (a targeter-RNA and an activator-RNA) that are complementary to one another, are covalently linked (directly, or by intervening nucleotides), and hybridize to form the double stranded RNA duplex (dsRNA duplex) of the protein-binding segment, thus resulting in a stem-loop structure. The targeter-RNA and the activator-RNA can be covalently linked via the 3′ end of the targeter-RNA and the 5′ end of the activator-RNA. Alternatively, targeter-RNA and the activator-RNA can be covalently linked via the 5′ end of the targeter-RNA and the 3′ end of the activator-RNA.

The term “activator-RNA” is used herein to mean a tracrRNA-like molecule of a double-molecule guide RNA. The term “targeter-RNA” is used herein to mean a crRNA-like molecule of a double-molecule guide RNA. The term “duplex-forming segment” is used herein to mean the stretch of nucleotides of an activator-RNA or a targeter-RNA that contributes to the formation of the dsRNA duplex by hybridizing to a stretch of nucleotides of a corresponding activator-RNA or targeter-RNA molecule. In other words, an activator-RNA comprises a duplex-forming segment that is complementary to the duplex-forming segment of the corresponding targeter-RNA. As such, an activator-RNA comprises a duplex-forming segment while a targeter-RNA comprises both a duplex-forming segment and the DNA-targeting segment of the guide RNA. Therefore, a double-molecule guide RNA can be comprised of any corresponding activator-RNA and targeter-RNA pair.

By “recombination” it is meant a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA. In some embodiments, the donor polynucleotide, a portion of the donor polynucleotide, a copy of the donor polynucleotide, or a portion of a copy of the donor polynucleotide integrates into the target DNA

By “non-homologous end joining (NHEJ)” it is meant the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a plant or an animal, such as a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The therapy will desirably be administered during the symptomatic stage of the disease, and in some cases after the symptomatic stage of the disease.

The terms “individual,” “subject,” and “patient,” are used interchangeably herein and refer to any plant or animal, such as a mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The phrase “consisting essentially of is meant herein to exclude anything that is not the specified active component or components of a system, or that is not the specified active portion or portions of a molecule.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the invention, which are, for clarity, described in the context of separate embodiments, may also be provided in combination in a single embodiment.

Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As has been stated in the introduction, the gene editing of eukaryotic cells has been advanced by the invention that the Type II CRISPR-Cas system could be used as a target- and replace-mechanism for making mutations in de nucleic acid of the target cell. The methods that thus far have been used to provide such eukaryotic cells with the Type II Cas endonuclease enzyme (Cas9) have used mainly viral vectors carrying the nucleic acid encoding the Cas9 enzyme. However, the Cas9 enzyme is relatively large and incorporation into a viral vector often leads to difficulties or may even be impossible. This is especially the case if the nucleic acid which needs to be inserted through gene editing with the Cas9 enzyme is also large.

The current inventors now have found that a lot of unwanted side effects occur when the nuclease, i.e. the Cas9, and the guide RNA are present as separate entities in the transformed or transfected cell. Especially the nuclease when unbound to gRNA seems to provide effects that can lead to toxicity and/or apoptosis of the cells. Apparently (see the below experimental evidence) the Cas9 enzyme is steered towards the incorrect target nucleic acid sequences, which may be caused because the guide RNA acts a-specifically with respect to the target DNA. Another possibility is that the nuclease recognizes or works together with pieces of DNA that are already present in the target cell and which thus would play the role of guide DNA, thereby ‘mis’guiding the nuclease. It has been shown that mutating the Streptococcus pyogenes Cas9 enzyme can lead to a reduction in this off-targeting effect (Kleinstiver et al., Nature, 2016 doi:10.1038/nature16526). However, in cases where it is advantageous to use wild-type enzymes or in which other enzymes than Cas9 from Streptococcus pyogenes are desirable other solutions should be sought. One of the possibilities is to prevent the nuclease to complex with other RNAs than the guide RNA with which it is desired to complex.

Such an effect may be achieved by overexpression of the gRNA with respect to (expression of) the nuclease. This can be achieved by providing the cell with a construct in which the gRNA is expressed under control of a strong constitutive promoter, while the Type II nuclease enzyme is expressed in a less abundant number or provided to the cell through transfection. However, since the nuclease and the gRNA will need time to find each other in the cell and form a complex, there still is the risk that off-target effects can occur from unbound nuclease enzyme.

One way to improve on this is by providing the nuclease-guide RNA complex to the cell in a complete manner. In this embodiment, the Type II enzyme and the gRNA are complexed outside the cell, any unbound enzyme and any unbound gRNA then preferably is removed from the solution, and the complexes are then transfected into the cell, e.g. by lipofection. In this case the presence of free nuclease enzyme or nuclease enzyme coupled to pieces of DNA that are endogenous to the cell to be transfected is minimalized.

However, in this embodiment it is still possible that nuclease enzyme and gDNA are separated and that free nuclease can be introduced into the cell where it can exert the deleterious effects. For this reason it is preferred that the nuclease is tightly connected to the guide RNA before the complex would enter the target cell. In the alternative, the prevention of the above-mentioned off-targeting may also be caused by assuring that the nuclease is complexed to the (correct) guide RNA before it exercises its function. A first possible embodiment in which this can be effected is first cross-linking the guide RNA with the nuclease in a cell or in vitro system in which both components are available.

It is preferred that the guide RNA will be cross-linked to the Cas9 protein or otherwise firmly attached to it to enable proper functioning of the Cas9-guide RNA complex. Such a cross-linking can be established by means known to the skilled person for crosslinking proteins to nucleic acids. One specific embodiment of such a cross-linking has been described by Saito and Matsura (1985, Acc. Chem. Res. 18:134-141) who described photoreactions with lysine and tryptophan to e.g. thymidine moieties on the nucleic acid. Such a coupling may be performed by irradiation with UV light. As is shown in the experimental part below this yields a still functional nuclease. Further, appropriate photo-cross-linking with p-benzoyl-L-phenylalanine (pBpa) has been described (Farrell, I. et al., Nature Meth. 2:377-384, 2005).With the use of this technique the protein to be cross-linked, in this case the nuclease enzyme, can be (site) specifically provided with the pBpa at any site of the protein, after which the nucleic acid may be crosslinked to the protein. Also, such a specific cross-linkable site provides for an excellent specificity for the photo-induced cross-linking reaction. Another possibility is to cross-link the nuclease protein with the RNA in a reaction using formaldehyde (Moller, K. et al., 1977, Eur. J. Biochem. 76:175-187). Other bifunctional reagents that may be employed in cross-linking reactions are diepoxybutan (Skold, 1981, Biochimie 63(1):53-60), trans-diaminedichloroplatinum (II) (Tukalo et al., 1987, Biochem. 26(16):5200-5208) and 1-ethyl-3(3-dimethylaminopropyl)carbodiimide (EDC). It would also be possible to first modify the RNA molecule (e.g. to contain adenosine moieties modified to contain a disulfide bond and an alkyl chain, where the disulfide bond can be reduced with a reactive thiol and then cross-linked to an amino acid via reaction with benzophenone. Also nucleosides with tethers ending in primary amines (which are e.g. commercially available) can be derivatized with cross-linking reagents containing isothiocyanate or succinimide ester functional groups. A nucleotide that is readily available for cross-linking is 4-thiouridine.

Also, in order to further avoid off-targeting it is preferred that a nuclease is used which is heterologous to the organism or cell which is targeted by the Cas9-guide RNA complex. A heterologous Cas9 enzyme can be provided by transforming an intended bacterial vector or cell with a Cas9 enzyme from a different source. Many Cas9 enzymes are nowadays known and an extensive list has e.g. been provided in WO 2015/071474, more particularly in SEQ ID Nos: 1 -800 of said document, which list is herein included by reference. Most preferred is the Cas9 enzyme from Campylobacter jejuni, since the Cas9 enzyme from this bacterium is relatively small (e.g. compared to the much used Cas9 enzyme of S. pyogenes) (Kim, E. et al., 2017, Nature Comm. 8:14500). Also the Cas9 ortholog from Staphylococcus aureus is more than 1 Kb shorter than the S. pyogenes Cas9 (Ran, F. et al., Nature. 520:186-191, 2015)

Notwithstanding the preference for smaller Type II Cas enzymes, the present invention is not critical in this respect and any Type II Cas nuclease, such as Cas9 that works together with a guide RNA may be used. The Cas9 enzyme preferably is derived from Pasteurella multocida, Streptococcus thermophilus, Streptococcus agalactiae, Streptococcus anginosus, Streptococcus bouis, Streptococcus canis, Streptococcus constellatus, Streptococcus dysgalactiae, Streptococcus equi, Streptococcus equinus, Streptococcus gallolyticus, Streptococcus infantarius, Streptococcus iniae, Streptococcus macacae, Streptococcus mitis, Streptococcus oxalis, Streptococcus gordonii, Streptococcus infantarius, Streptococcus macedonicus, Streptococcus parasanguinis, Streptococcus pasteurianus, Streptococcus pseudoporcinus, Streptococcus ratti, Streptococcus salivarius, Streptococcus sanguinis, Streptococcus suis, Streptococcus pyogenes, Streptococcus mutans, Streptococcus vestibularis, Pediococcus acidilactici, Staphylococcus aureaus, Staphylococcus lugdunensis, Staphylococcus pseudintermedius, Staphylococcus simulans, Escherichia coli, Neisseria bacilliformis, Neisseria cinerea, Neisseria flauescens, Neisseria lactamica, Neisseria meningitides, Neisseria wadsworthii, Listeria innocua, Francisella nouicida, Campylobacter jejuni, Campylobacter coli, Campylobacter lari, Helicobacter canadensis, Helicobacter cinaedi, Lactobacillus animalis, Lactobacillus farciminis, Lactobacillus buchneri, Lactobacillus casei, Lactobacillus coryniformis, Lactobaci/lus farciminis, Lactobacillus fermentum, Lactobacillus forum, Lactobacillus gasseri, Lactobacillus hominis, Lactobacillus iners, Lactobacillus jensenii, Lactobacillus johnsonii, Lactobacillus mucosae, Lactobacillus paracasei, Lactobacillus pentosus, Lactobacillus plantarum, Lactobacillus rhamnosus, Lactobacillus ruminis, Lactobacillus salivarius, Lactobacillus sanfranciscensis, Lactobaci/lus uersmoldensis, Legionella pneumophila, Listeria monocytogenes, Acidaminococcus intestine, Acidothermus cellulolyticus, Acidouorax ebreus, Actinobacillus minor, Actinobacillus pleuropneumoniae, Actinobacillus succinogenes, Actinobacillus suis, Actinomyces coleocanis, Actinomyces georgiae, Actinomyces naeslundii, Actinomyces turicensis, Acidouorax auenae, Akkermansia muciniphila, Alicycliphilus denitrificans, Alicyclobacillus hesperidum, Aminomonas pauciuorans, Anaerococcus tetradius, Anaerophaga thermohalophila, Bacillus cereus, Bacillus smithii, Bacillus thuringiensis, Bacteroides coprophilus, Bacteroides coprosuis, Bacteroides dorei, Bacteroides eggerthii, Bacteroides faecis, Bacteroides fluxus, Bacteroides fragilis, Bacteroides nordii, Bacteroides uniformis, Bacteroides uulgatus, Barnesiella intestinihominis, Bergeyella zoohelcum, Bifidobacterium bifidum, Bifidobacterium dentium, Bifidobacterium longum, Breuibacillus laterosporus, Caenispirillum salinarum, Capnocytophaga gingivalis, Capnocytophaga canimorsus, Capnocytophaga sputigena, Catellicoccus marimammalium, Catenibacterium mitsuokai, Clostridium perfringens, Clostridium spiroforme, Coprococcus catus, Coriobacterium glomerans, Corynebacterium accolens, Corynebacterium diphtheria, Dinoroseobacter shibae, Dorea longicatena, Dolosigranulum pigrum, Elusimicrobium minutum, Enterococcus faecalis, Enterococcus faecium, Enterococcus hirae, Enterococcus itaficus, Eubacterium dolichum, Eubacterium rectale, Eubacterium uentriosum, Eubacterium yurii, Facklamia hominis, Fibrobacter succinogenes, Filifactor alocis, Finegoldia magna, Flavobacterium branchiophilum, Flavobacterium columnare, Flavobacterium psychrophilum, Fluuiicola taffensis, Francisella tgularensis, Fructobacillus fructosus, Fusobacterium nucleatum, Gardnerella vaginalis, Gemella haemolysans, Gemella morbillorum, Gluconacetobacter diazotrophicus, Gordonibacter pamelaeae, Haemophilus parainfluenzae, Haemophilus sputorum Helcococcus kunzii, Helicobacter mustelae, lndibacter alkaliphilus, lgnauibacterium album, llyobacter polytropus, Joostella marina, Kordia algicides, Leuconostoc gelidum, Methylosinus trichosporium, Mucilaginibacter paludis, Myroides injenensis, Myroides odoratus, Mobiluncus curtisii, Mobiluncus mulieris, Mycoplasma canis, Mycoplasma gallisepticium, Mycoplasma mobile, Mycoplasma ouipneumoniae, Mycoplasma synouiae, Niabella soli, Nitratifractor salsuginis, Nitrobacter hamburgensis, Odoribacter laneus, Oenococcus kitaharae, Ornithobacterium rhinotracheale, Parabacteroides johnsonii, Parasutterella excrementihominis, Paruibaculum lauamentiuorans, Phascolarctobacterium succinatutens, Planococcus antarcticus, Prevotella biuia, Prevotella buccae, Prevotella buccalis, Prevotella denticola, Prevotella histicola, Prevotella intermedia, Prevotella micans, Prevotella oralis, Prevotella nigrescens, Prevotella ruminicola, Prevotella stercorea, Prevotella tannerae, Prevotella timonensis, Prevotella ueroralis, Ralstonia syzygii, Rhodopseudomonas palustris, Rhodospirillum rubrum, Riemerella anatipestifer, Roseburia intestinalis, Ruminococcus albis, Ruminococcus lactaris, Scardouia inopinata, Scardouia wiggsiae, Solobacterium moorei, Sphaerochaeta globus, Sphingobacterium spiritiuorum, Streptobacillus moniliformis, Sutterella wadsworthensis, Treponema denticola, Tistrella mobilis, Veillonella atypica, Veillonella paruula, Weeksella uirosa, Wolinella succinogenes or Zunongwangia profunda.

Preferably the Cas protein is derived from S. pyogenes, S. thermophilus, S. mutans, C. jejuni, F. nouicida, P. multocida or N. meningitides.

Preferably, the guide RNA that is used in the present invention comprises a CRISPR sequence. Such a CRISPR sequence may be a CRISPR sequence that is derived from bacterial or archaeal origin, but it may also be a CRISPR that is derived from other organisms, such as the CRISPR sequences disclosed in co-pending application PCT/NL2015/050438. The guide RNA further will comprise a DNA-targeting sequence as defined above and a protospacer adjacent motif (PAM) sequence.

Cross-linking of the nuclease with the guide RNA may be accomplished in a vector cell, especially the vector cell in which the heterologous nuclease has been expressed and in which also the guide RNA is expressed either on the same construct as the nuclease or by any other means. Cross-linking may also take place in vitro, in a composition comprising the nuclease, where the nuclease is isolated from a cell that produces said nuclease, or as a readily available preparation from a commercial source. Then the guide RNA is added to the composition and cross-linking is performed, such as cross-linking through UV radiation or chemical reaction.

If cross-linking is performed in a living cell or biological vector (such as a virus particle) then advantageously it should be established that at little as possible and preferably no unbound nuclease is left in said cell or vector. This can be advantageously accomplished by starting the cross-linking reaction with a higher concentration of guide RNA. Excess guide RNA will not lead to off-targeting effects.

If cross-linking is performed in vitro the cross-linked complex may be taken up by a vector for delivering it to a target cell. The cross-linked complex may also be directly delivered into a target cell by any of the known transfection methods, such as electroporation and the like. It is also possible to use systems or methods that are known to be able to transfer macromolecules into a cell, such as liposomes and/or cationic amphiphilic compounds.

The Cas9 enzyme exhibits nuclease activity that cleaves target DNA at a target DNA sequence defined by the region of complementarity between the guide RNA and the target DNA. Then site-specific cleavage of the target DNA occurs at locations determined by both (i) base-pairing complementarity between the guide RNA and the target DNA; and (ii) a short motif [referred to as the protospacer adjacent motif (PAM)] in the target DNA. In some embodiments (e.g., when Cas9 from S. pyogenes is used), the PAM sequence of the non-complementary strand is 5′-XGG-3′, where X is any DNA nucleotide and X is immediately 3′ of the target sequence of the non-complementary strand of the target DNA. As such, the PAM sequence of the complementary strand is 5′-CCY-3′, where Y is any DNA nucleotide and Y is immediately 5′ of the target sequence of the complementary strand of the target DNA (where the PAM of the non-complementary strand is 5′-GGG-3′ and the PAM of the complementary strand is 5′-CCC-3′). In some such embodiments, X and Y can be complementary and the X-Y base pair can be any basepair (e.g., X=C and Y=G; X=G and Y=C; X=A and Y=T, X=T and Y=A).

In some cases, different Cas9 proteins (i.e., Cas9 proteins from various species) may be advantageous to use in the various provided methods in order to capitalize on various enzymatic characteristics of the different Cas9 proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

Cas9 proteins from various species, such as the species listed above (see further SEQ ID NOs: 1-800 of WO 2015/071474) may require different PAM sequences in the target DNA. Thus, for a particular Cas9 protein of choice, the PAM sequence requirement may be different than the 5′-XGG-3′ sequence described above. For example, for Campylobacter jejuni PAM sequence NNNNACA; for P. multocida PAM sequences GNNNCNNA or NNNNC; for F. nouicida PAM sequence NG; for S. thermophiles a PAM sequence NNAAAAW; for L. innocua a PAM sequence NGG; and for S. dysgalactiae PAM sequence NGG may be needed (see e.g. Fonfara, I. et al., Nucl. Acids Res. 42:2577-2590, 2014).

The nuclease activity cleaves target DNA to produce double strand breaks. These breaks are then repaired by the cell in one of two ways: non-homologous end joining, and homology-directed repair. In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. In some cases, a target DNA is contacted with a donor polynucleotide. In some cases, a donor polynucleotide is introduced into a cell. The modifications of the target DNA due to NHEJ and/or homology-directed repair lead to, for example, gene correction, gene replacement, gene tagging, transgene insertion, nucleotide deletion, gene disruption, gene mutation, sequence replacement, etc.

In some embodiments, the Cas9 protein comprises a heterologous sequence which can provide for subcellular localization of the site-directed modifying polypeptide (e.g., a nuclear localization signal (NLS) for targeting to the nucleus; a mitochondrial localization signal for targeting to the mitochondria; a chloroplast localization signal for targeting to a chloroplast; a ER retention signal; and the like). In some embodiments, a heterologous sequence can provide a tag for ease of tracking or purification (e.g., a fluorescent protein, e.g., green fluorescent protein (GFP), YFP, RFP, CFP, mCherry, tdTomato, and the like; a his tag, e.g., a 6× His tag; a hemagglutinin (HA) tag; a FLAG tag; a Myc tag; and the like). In some embodiments, the heterologous sequence can provide for increased or decreased stability.

The Cas9 gene can be codon-optimized. This type of optimization is known in the art and entails the mutation of foreign-derived DNA to mimic the codon preferences of the intended vector or host organism or cell while encoding the same protein. Thus, the codons are changed, but the encoded protein remains unchanged. For example, if the intended target cell was a human cell, a human codon-optimized Cas9 (or variant, e.g., enzymatically inactive variant) would be a suitable enzyme. While codon optimization is not required, it is acceptable and may be preferable in certain cases. Polyadenylation signals can also be chosen to optimize expression in the intended host.

In some of the above applications, the methods may be employed to induce DNA cleavage, DNA modification, and/or transcriptional modulation in mitotic or post-mitotic cells in vivo and/or ex vivo and/or in vitro (e.g., to produce genetically modified cells that can be reintroduced into an individual).

Because the guide RNA provides specificity by hybridizing to target DNA, a target cell of interest in the disclosed methods may include a cell from any eukaryotic organism (e.g. a cell of a single-cell eukaryotic organism, a plant cell, an algal cell, e.g., Botryococcus braunii, Chlamydomonas reinhardtii, Nannochloropsis gaditana, Chorella pyrenoidosa, Sargassum patens and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a primate, a cell from a human, etc.).

Any type of cell may be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Human embryo's and cells derived from human embryos are excluded from the present invention. Cells may be from established cell lines or they may be primary cells, where “primary cells”, “primary cell lines”, and “primary cultures” are used interchangeably herein to refer to cells and cells cultures that have been derived from a and allowed to grow in vitro for a limited number of passages of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro. Target cells are in many embodiments unicellular organisms, or are grown in culture.

If the cells are primary cells, they may be harvested from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

In some embodiments, a method involves contacting a target DNA or introducing into a cell (or a population of cells) the cross-linked complex of a guide RNA and a Cas9 enzyme and/or a donor polynucleotide. Contacting the cells with a cross-linked complex of a guide RNA and Cas9 enzyme and/or donor polynucleotide may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which the cells are responsive.

Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Conditions that promote the survival of cells are typically permissive of non-homologous end joining and homology-directed repair. In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by the Cas9 enzyme. The donor polynucleotide will contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide. The donor sequence may comprise certain sequence differences as compared to the genomic sequence, e.g. restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequence differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by the bacterial vectors, as described above for nucleic acids encoding a guide RNA and/or site-directed modifying polypeptide and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans Animal models, particularly small mammals (e.g. mouse, rat, guinea pig, hamster, lagomorpha (e.g., rabbit), etc.) may be used for experimental investigations. Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5,385,582); by bolus injection, e.g. by a syringe, e.g. into a joint; by continuous infusion, e.g. by cannulation, e.g. with convection (see e.g. US Application No. 20070254842); or by implanting a device upon which the cells have been reversibly affixed (see e.g. US Application Nos. 20080081064 and 20090196903). Cells may also be introduced into an embryo (e.g., a blastocyst) for the purpose of generating a transgenic animal (e.g., a transgenic mouse).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In other aspects of the disclosure, the guide RNA and/or site-directed modifying polypeptide and/or donor polynucleotide are employed to modify cellular DNA in vivo, again for purposes such as gene therapy, e.g. to treat a disease or as an antiviral, anti-pathogenic, or anticancer therapeutic, for the production of genetically modified organisms in agriculture, or for biological research. In these in vivo embodiments, the Cas9 enzyme cross-linked to a guide RNA and/or a donor polynucleotide are administered directly to the individual. When these components are administered in addition to the administration through a vector as described hereinbefore, they may be administered by any of a number of well-known methods in the art for the administration of peptides, small molecules and nucleic acids to a subject. Such a peptide or nucleic acid component can be incorporated into a variety of formulations. More particularly, the guide RNA—nuclease complex and/or donor polynucleotide can be formulated into pharmaceutical compositions by combination with appropriate pharmaceutically acceptable carriers or diluents.

Accordingly, the gene-editing method as discussed above may be used to delete nucleic acid material from a target DNA sequence, to remove disease-causing trinucleotide repeat sequences in neurons, to create gene knockouts and mutations as disease models in research, etc. by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material into a locus of choice in the target DNA.

Alternatively, if the cross-linked complex of a guide RNA and a Cas9 enzyme is co-administered to cells with a donor polynucleotide sequence that includes at least a segment with homology to the target DNA sequence, e.g. by providing all components within one and the same vector or administration vehicle, the subject methods may be used to add, i.e. insert or replace, nucleic acid material to a target DNA sequence (e.g. to “knock in” a nucleic acid that encodes for a protein, an siRNA, an miRNA, etc.), to add a tag (e.g., 6× His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, etc.), to modify a nucleic acid sequence (e.g., introduce a mutation), and the like. As such, a complex comprising a guide RNA and a Cas9 enzyme is useful in any in vitro or in vivo application in which it is desirable to modify DNA in a site-specific, i.e. “targeted”, way, for example gene knock-out, gene knock-in, gene editing, gene tagging, sequence replacement, etc., as used in, for example, gene therapy, e.g. to treat a disease or as an antiviral, anti-pathogenic, or anticancer therapeutic, the production of genetically modified organisms in agriculture, the large scale production of proteins by cells for therapeutic, diagnostic, or research purposes, the induction of iPS cells, biological research, the targeting of genes of pathogens for deletion or replacement, etc.

The invention is illustrated in the below examples, which are merely illustrative and not meant to limit the invention as described herein.

### EXAMPLES

The requirement in genome editing of the guide RNA to be delivered bound to Cas9 without its ability to be released is obtained from the following experimental evidence. We firstly examined the biology of Cas9 and found it behaved as a virulence factor and after that the mechanism by which Cas9 was able to damage DNA without a guide RNA. Finally we showed by proof of concept that off-targeting can be reduced to a minimum when the guide RNA is bound to Cas9 without the ability to be released.

### Example 1

### Campylobacter jejuni Cas9 is a Virulence Factor and able to Induce Cell Death

Campylobacter jejuni wild-type strains GB2, GB11, GB19, NCTC11168 and their Δcas9 (csnl) mutant variants were studied; A) adhesion onto; B) invasion into; and C) translocation across human Caco-2 cells. Colony forming units (CFU) were calculated per ml. Data are expressed as the standard error of the mean (SEM) of three independent experiments, each performed in duplicate; D) Cytotoxic effects of GB2, GB11, GB19, NCTC11168 and their Δcas9 (csn1) mutant variants on HT-29 cells were measured at OD490nm visualizing the lactate dehydrogenase (LDH) release in cell culture supernatants. The data are expressed as the SEM of triplicate determinations performed in triplicate. Significant differences between wild-type and Δcas9 (csnl) mutants were observed. In the adhesion and invasion assay, this significance was *p<0.05 and in the translocation and cytotoxicity assays *p<0.01 using a paired t-test between wild-type and Δcas9 (csnl) mutants, respectively. Notable, the NCTC11168 isolate and its cas9 mutant were not able to translocate. Material and method details and Figures see Louwen et al., EUJCMID 2012.

### Example 2

### Cas9 Positive Campylobacter jejuni Isolates damage Eukaryotic Cells

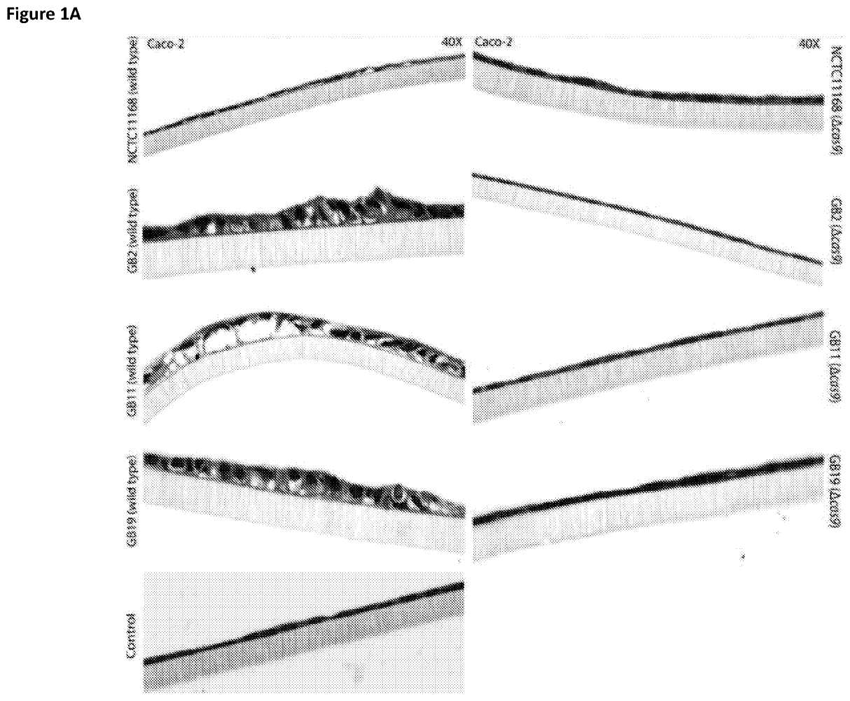

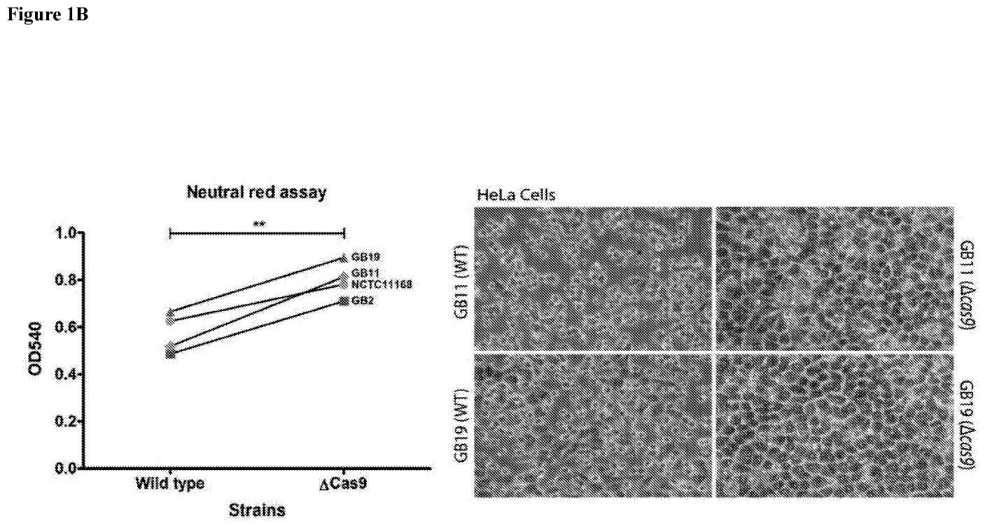

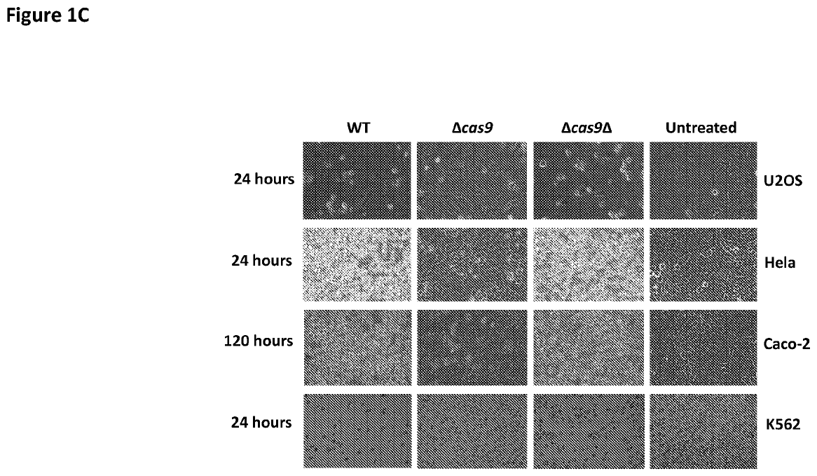

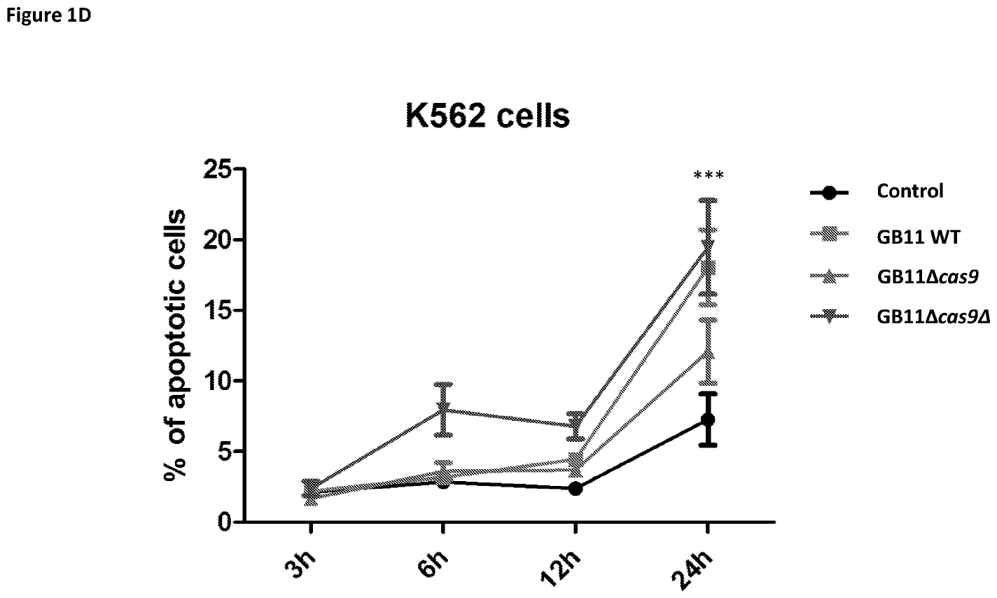

A) Caco-2 intestinal epithelial cells were seeded onto Transwell filters at 4×105 cells/filter (5-pm pore size, 1.13 cm2; Costar, Corning Inc., Corning, New York, USA) and allowed to differentiate and form tight junctions for 19 days. After 7-10 days of culture, the transepithelial electrical resistance (PEER) was >1,000 Ω/cm2, indicating the presence of an intact epithelial monolayer. Campylobacter jejuni isolates were added at a multiplicity of infection (MOI) of 10 to the apical surface of the Transwell filter at day 19. After 24 hours, Transwells were rinsed with PBS at 37° C. and fixated with 4% paraformaldehyde for 1 hour. Transwells were washed in 70% ethanol and dehydrated in 70% ethanol (2×15 minutes), 96% ethanol (2×20 minutes), 100% ethanol (1×10 minutes and 2×20 minutes) and 1 butanol (1×20 minutes and 2×30 minutes). Membranes were embedded in paraffin (Sigma-Aldrich, Zwijndrecht, The Netherlands) and stored at room temperature until sectioned. 5 ₁1M-thick slides were deparaffinated in xylene (Sigma-Aldrich, Zwijndrecht, The Netherlands) and hydrated in ethanol (100%, 96%, 90%, 80%, 70%, 50%) (Sigma-Aldrich, Zwijndrecht, The Netherlands) and then rinsed in H20. Transwell coupes were stained with HE staining (Sigma-Aldrich, Zwijndrecht, The Netherlands) and analysed using the phase contrast IX51 microscope (Olympus, Leiderdorp, The Netherlands) revealing that strains that harbor Cas9 (11168, GB11, GB2 and GB19) causes swelling of the Caco-2 intestinal epithelial cells an indication that these cells are experiencing damage, whereas this phenomenon was absent in their Δcas9 mutants or uninfected cells (FIG. 1A). The cell viability of HELA cells controls and of Campylobacter jejuni GB2, GB11, GB19, NCTC11168 and their isogenic Δcas9 variants overnight-was determined using the neutral red assay. Neutral red was measured using the in vitro toxicology assay kit (Sigma-Aldrich, Zwijndrecht, The Netherlands). Incubation was performed in DMEM medium without phenol red (Invitrogen, Breda The Netherlands) and only containing 1× NEAA (Lonza, Verviers, Belgium). Protocol was followed according the manufacturer. The experiments were repeated three times. Neutral red assay demonstrated that HELA cells infected with Cas9 positive Campylobacter jejuni isolates lost viability and became apoptotic/necrotic (FIG. 1B). U205, HELA, Caco-2 and K562 cells were grown to 70-90% confluence in a 12 well plate in DMEM medium containing 10% FBS and 1X non-essential amino acids. Campylobacter jejuni strain GB 11, its isogenic Δcas9 variant or its Δcas9Δ complemented variant was resuspended in the above described cell culture medium and inoculated at a MOI of 100 onto the eukaryotic cells. After 24, 48, 72, 96 and 120 hours of infection microscopic pictures were taken using the phase contrast IX51 microscope (Olympus, Leiderdorp, The Netherlands) at a 20x magnification in bright field showing that GB11 and its Δcas9Δ complemented variant significantly killed the eukaryotic cells, although the time of killing was cell line dependent. The severe killing was not observed for the GB11Δcas9 variant and in the uninfected cells demonstrating that Cas9 is associated with severe cell damage (FIG. 1C). K562 cells were grown to 70-90% confluence in a 12 well plate in DMEM medium containing 10% FBS and 1X non-essential amino acids. Campylobacter jejuni strain GB11, its isogenic Δcas9 variant or its Δcas9Δ complemented variant was resuspended in the above described cell culture medium and inoculated at a MOI of 100 onto the eukaryotic cells. After 24 hours a cell death assay (Thermofisher scientific, Breda, The Netherlands) was performed showing that GB11 and its Δcas9Δ complemented variant significantly killed the K562 cells. The severe killing was not observed for the GB11Δcas9 variant and in the uninfected cells demonstrating that Cas9 is associated with severe cell damage (FIG. 1D)

### Example 3

### Cas9 Induces DNA Damage and Apoptosis Pathways

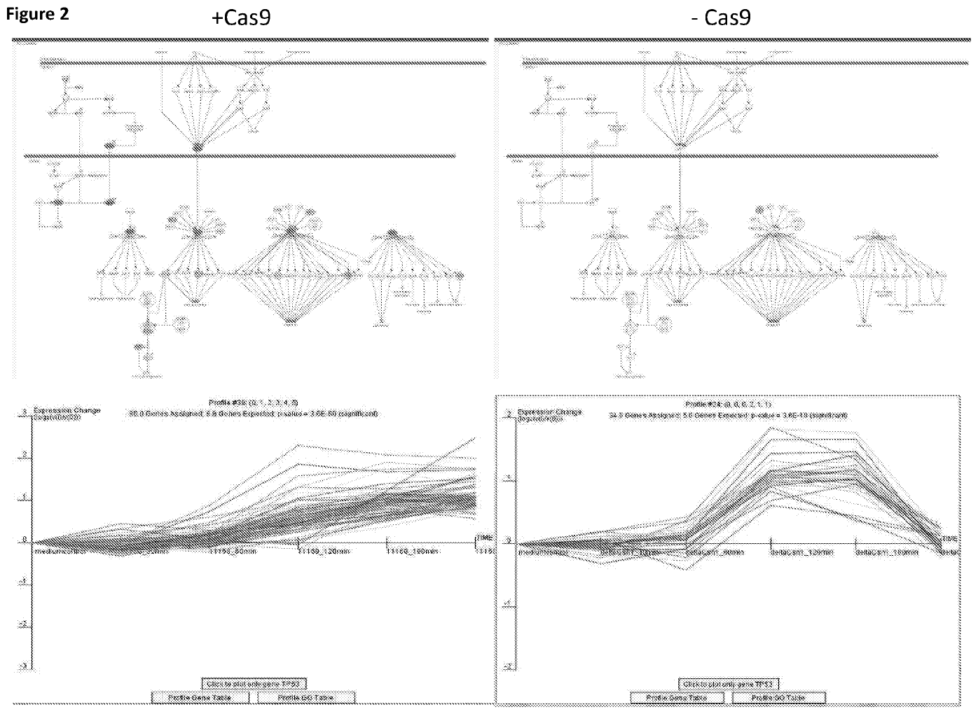

Caco-2 intestinal epithelial cells were incubated with wild type or a Δcas9 Campylobacter jejuni strain and RNA was extracted using Trizol (Sigma-Aldrich, Zwijndrecht, The Netherlands) and hybridized to human whole-genome gene expression microarrays (Affymetrix, Charleroi, Belgium). To capture the induction or silencing of different pathways at specific time points, RNA was extracted at five time points within four hours and for each time point three replicates were obtained. The time points were rationally chosen based on earlier microscopic analysis of Caco-2 infection by different Campylobacter jejuni bacteria (Louwen et al., IAI, 2012). From this assay it became evident that Cas9 alters DNA and chromatin dynamics, and gene expression, via a DNA damage mechanism(s) that is perceived by ATM and signaled via p53-MAPK pathways leading to apoptosis, but not in the Δcas9 Campylobacter jejuni strain (FIG. 2).

### Example 4

### Cas9 is Secreted by Campylobacter jejuni into Eukaryotic Cells

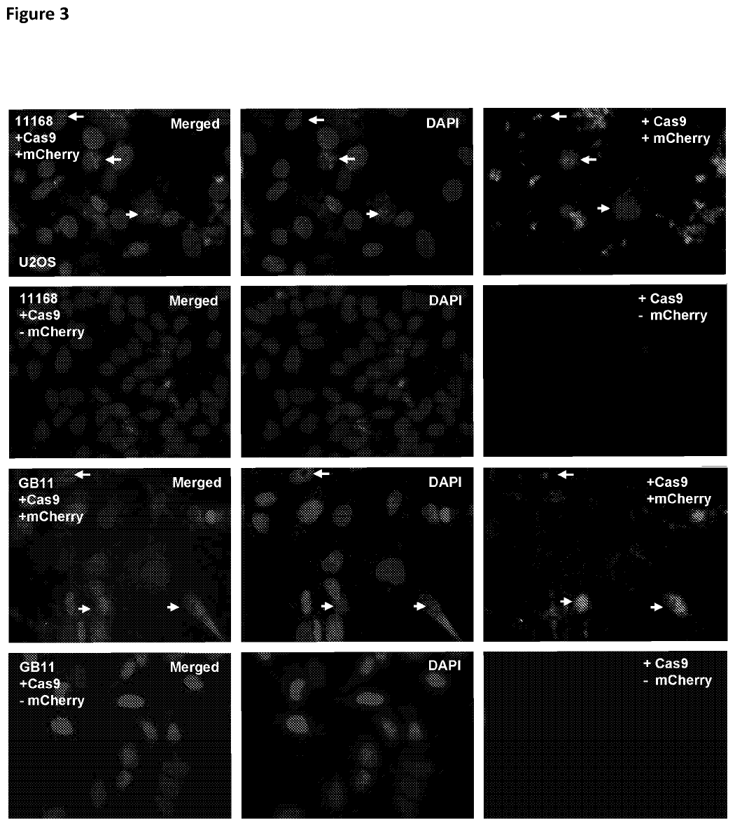

A Campylobacter jejuni strain NCTC11168 with Cas9 fused to mCherry was kindly provided by Dr. Cynthia Sharma, Wurzburg university, Wurzburg, Germany. Cas9 fused to mCherry was cloned into a different Campylobacter jejuni strain named GB11 by homologues recombination. U2OS bone marrow epithelial cells grown on a 2-well chamber slide (Greiner Bio-one, Alphen aan den Rijn, The Netherlands) were infected with a MOI of 100 with Campylobacter jejuni strain NCTC11168 or GB11 that harbored a Cas9 fused to mCherry. After overnight infection, U2OS cells were washed 3 times with pre-warmed HBSS at 37 OC and fixated with 4% paraformaldehyde (Sigma-Aldrich, Zwijndrecht, The Netherlands). After dehydration with 70% and 100% ethanol (Sigma-Aldrich, Zwijndrecht, The Netherlands) for 1 minute, the nuclei were stained with DAPI (Sigma-Aldrich, Zwijndrecht, The Netherlands), preserved in mounting medium (Sigma-Aldrich, Zwijndrecht, The Netherlands) and a cover slip was added on top and the secretion of mCherry-Cas9 was judged by using the IX51 phase contrast fluorescence microscopy (Olympus, Leiderdorp, The Netherlands) . It was observed that mCherry-Cas9 localized into the cytoplasm, nucleus and nucleolus of the U2OS cells when localized in the nucleus the cells were showing apoptosis and nuclei degradation (FIG. 3).

### Example 5

### Cas9 Localises into the Nucleus and Nucleolus

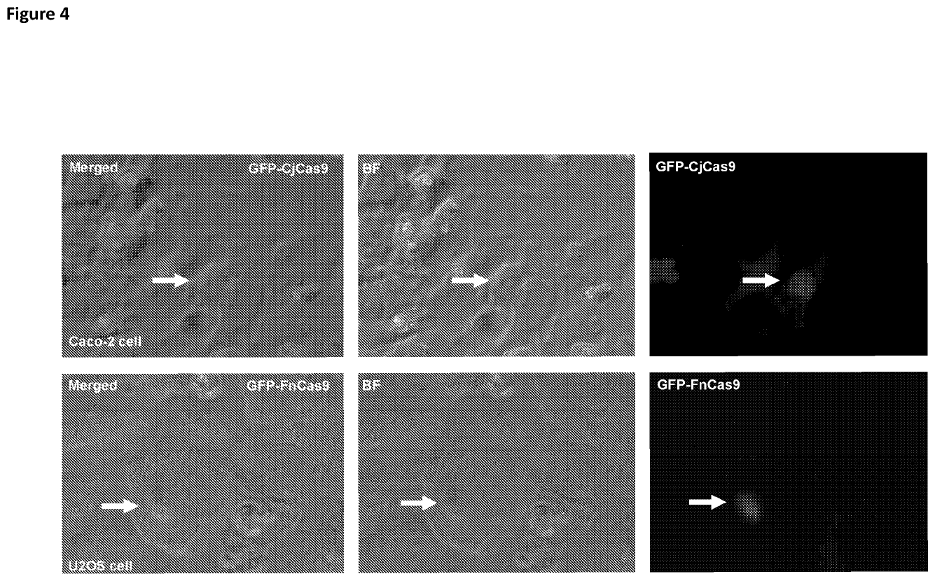

The coding sequences of the Cas9 proteins of Campylobacter jejuni GB11 and Francisella nouicida were cloned into pEGFP-C1 vector (Kindly provided by Prof. Anna Akhmanova UMC utrecht, Utrecht, The Netherlands) to express the fusion proteins in eukaryotic cells. In pEGFP-C1, EGFP-Cas9 and Cas9-EGFP fusion proteins are expressed under the control of the CMV IE promoter, respectively. Genomic DNA was isolated using the QlAamp DNA tissue stool kit (Qiagen, Venlo, The Netherlands), and amplification was performed in a 50 pl total volume, comprising 10 ng chromosomal DNA, 50 pmol of each primer, 20 mM dNTP (Thermoscientific, Breda, The Netherlands), 5 μl of 10× Supertaq buffer (Sphaero Q, Gorinchem, The Netherlands) and 2 units of Supertaq (Sphaero Q, Gorinchem, The Netherlands). PCR assays were performed using a Biomed Thermal Cycler System 9700 (Applied Biosystems, Bleiswijk, The Netherlands) with a program consisting of 30 cycles of 30 sec. at 95° C., 30 sec. at 55° C. and 3:30 min at 72° C. DNA digestion (Restriction enzymes, New England Biolabs, Leiden, The Netherlands), ligation (T4 DNA ligase, Thermofisher scientific, Breda, The Netherlands), purification and agarose gel electrophoresis were performed according to the manufacturer's protocols. Primer pairs that included the correct corresponding restriction enzymes for cloning used in this study are listed in Table 1. The resulting constructs were electroporated at 2.5 kV, 200 Ω, 25 pF into Escherichia coli Top10 cells (Invitrogen, Breda, The Netherlands), resuspended in 37° C. pre-warmed SOC medium (Invitrogen, Breda, The Netherlands), and allowed to recover by gentle shaking at 37° C. After recovery, 100 μl was plated onto a Lysogeny broth (LB; Becton Dickinson, Drachten, The Netherlands) agar plate containing 50 μg/ml Kanamycin (Sigma-Aldrich, Zwijndrecht, The Netherlands) for selection. Positive colonies were grown for plasmid DNA isolation (Fermentas, IJsselstein, The Netherlands) and subjected to restriction digestion (EcoRI, New England Biolabs, Leiden, The Netherlands) to confirm the integrity of the constructs. HEK293T and U2OS cells were maintained in Dulbecco's modified Eagle's medium (DMEM) (Invitrogen, Breda, The Netherlands) supplemented with 10% fetal bovine serum (FBS) (Invitrogen, Breda, The Netherlands), 100 U/ml penicillin, 100 μg/ml streptomycin and 1% nonessential amino acids (NEAA) (Invitrogen, Breda, The Netherlands). The cells were cultured in a 75-cm2 flask (Greiner Bio-one, Alphen aan den Rijn, The Netherlands) at 37° C. and 5% CO2 in a humidified air incubator. Cells (U2OS and HEK293T) were transiently transfected with plasmid DNA using X-tremeGENE HP DNA Transfection Reagent (Roche Applied Science, Almere, The Netherlands), according to the manufacturer's protocols. Cas9 of Campylobacter jejuni or Francisella nouicida fused to GFP were found to localize into the nucleus and/or nucleolus(Figure 4).

### Example 6

### Westernblot Analysis Demonstrates that Cas9 Localizes in the Nucleus/Nucleolus

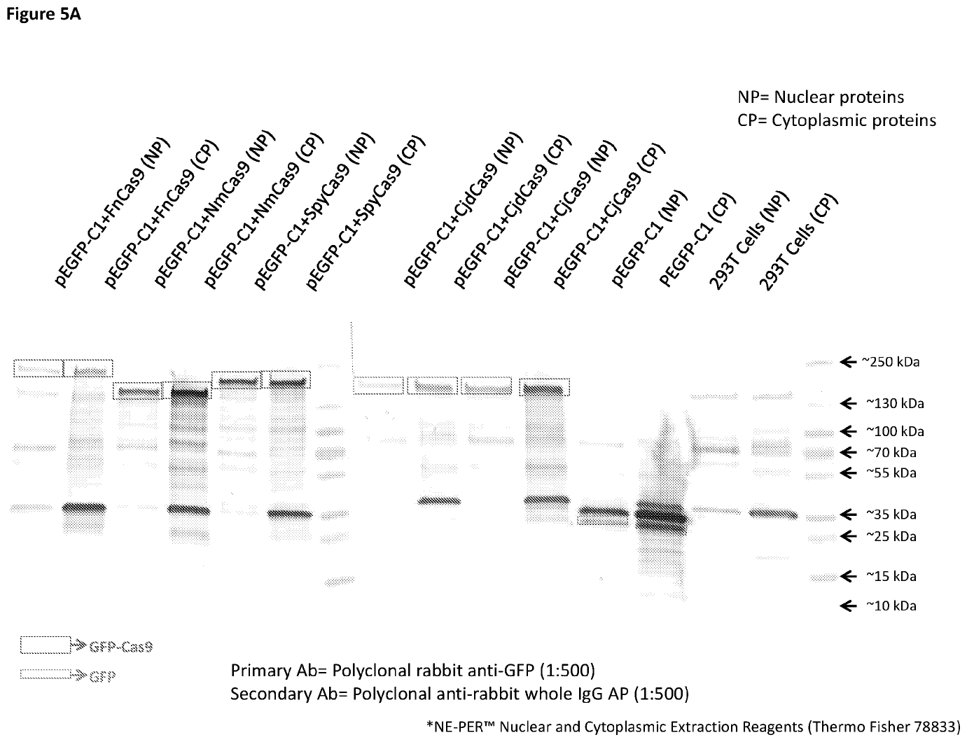

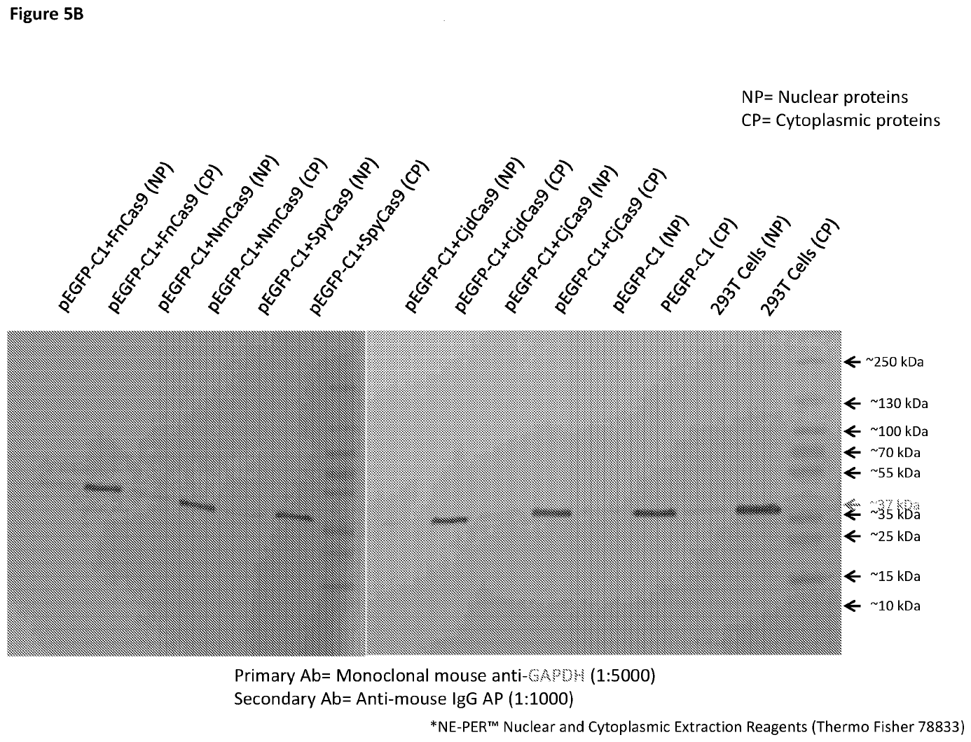

HEK293T eukaryotic cells were transfected with pEGFP-C1, pEGFP-C1-CjCas9, FnCas9, SpyCas9, NmCas9 or were left untreated. Cloning of the used Cas9 from Streptococcus pyogenes and Neisseria meningitidis occurred as described above (Example 5), but with different primers (Table 1). After 24 hours cells were harvested and treated according the NE-PER® Nuclear and Cytoplasmic Extraction Reagents (Thermo Fisher Scientific, Breda, The Netherlands). Protein fractions were loaded on a 4-20% gradient gel (Biorad, Veenendaal, The Netherlands). After separation proteins were transferred to a Hybond-P membrane (Amersham Biosciences, Roosendaal, The Netherlands) by electro-transfer. The membrane was the stained with the primary Polyclonal rabbit anti-GFP (1:500) (Genway, San Diego, USA) antibody or anti-GAPDH (1:5000) (Abeam, Cambridge, United Kingdom and the secondary polyclonal anti-rabbit whole IgG AP (1:500) or anti-mouse IgG AP (1:1000) to visualize the bacterial Cas9 proteins fused to GFP and GAPDH , respectively. The nuclear and cytoplasmic stainings demonstrated the localization of Campylobacter jejuni, Francisella nouicida, Streptococcus pyogenes and Neisseria meningitidis Cas9 into the nucleus of eukaryotic cells (FIG. 5A) and that the separation of the nuclear from the cytoplasmic fraction was successful (FIG. 5B).

### Example 7

### Cas9 Positive Campylobacter jejuni Isolates induce DNA Damage in Eukaryotic Cells

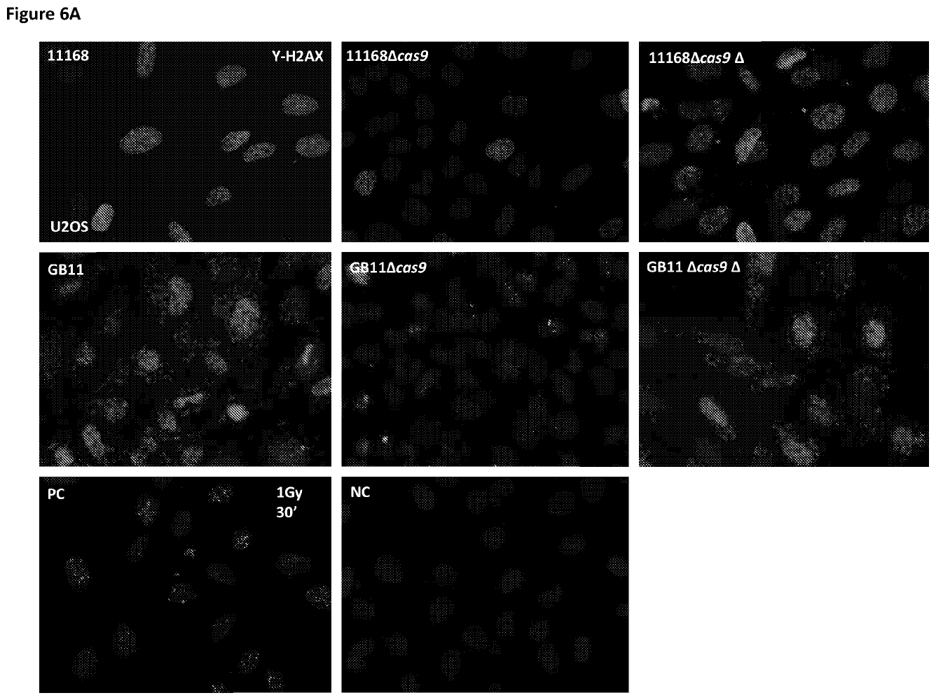

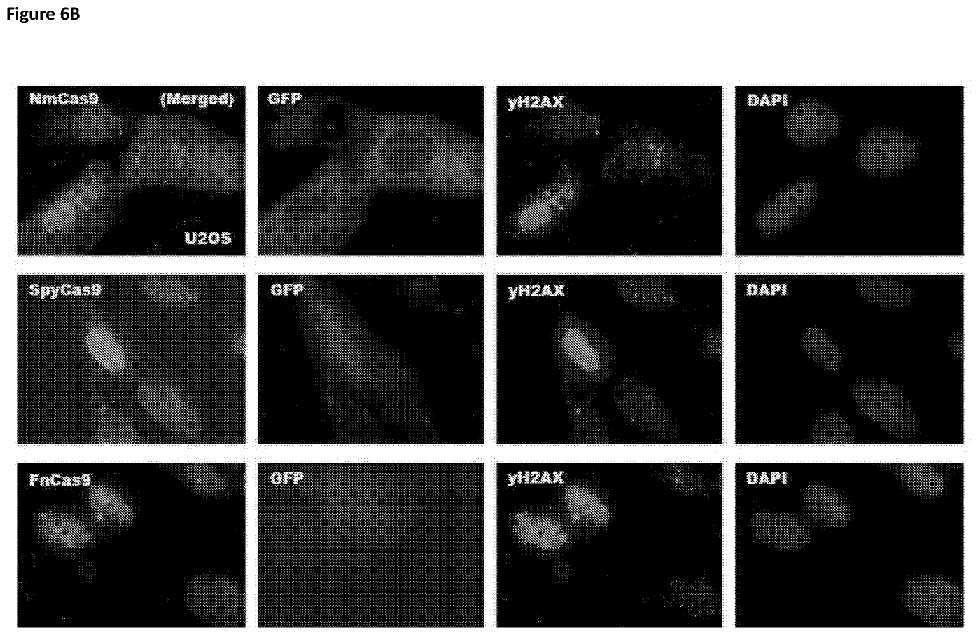

U2OS bone marrow epithelial cells were grown to 40% to 50% confluence on chamber slides (Greiner Bio-one, Alphen aan den Rijn, The Netherlands), and Campylobacter jejuni was inoculated at an MOI of 100. After 6 hours the U2OS cells were washed three times with room temperature HBSS and fixated with 4% paraformaldehyde (Sigma-Aldrich, Zwijndrecht, The Netherlands) and then permeabilized for 20 min with 0.1% HBSS—Triton X-100 solution (Sigma-Aldrich, Zwijndrecht, The Netherlands) and background antibody binding was blocked with block buffer (1% fetal bovine serum, 1% Tween-20 (Sigma-Aldrich, Zwijndrecht, The Netherlands), HBSS). Slides were then incubated for one hour with the respective primary antibody for y-H2AX at a 1:100 dilution in block buffer. The appropriate secondary antibodies from the IgG class (H+L), A594 labeled (Molecular Probes, Bleiswijk, The Netherlands), providing a red stain was used to detect y-H2AX thereby revealing that Campylobacter jejuni stained with anti-Campylobacter FITC (Genway, San Diego, USA) label (green) isolates with Cas9 are able to induce severe DNA damage after 6 hours in the eukaryotic U2OS cells. As a positive control for y-H2AX staining U2OS cells radiated with 1 Gy and fixated 30 minutes after radiation were taken along. Untreated U2OS cells were used as a negative control (FIG. 6A). Transfecting the U2OS cells with the plasmids pEGFPCas9 with Cas9 obtained from Francisella nouicida, Neisseria meningitidis or Streptococcus pyogenes showed that these Cas9 proteins are also able to induce severe DNA damage as visualized by y-H2AX staining (FIG. 6B).

### Example 8

### Campylobacter jejuni Cas9 alone induces DNA Damage in Eukaryotic Cells

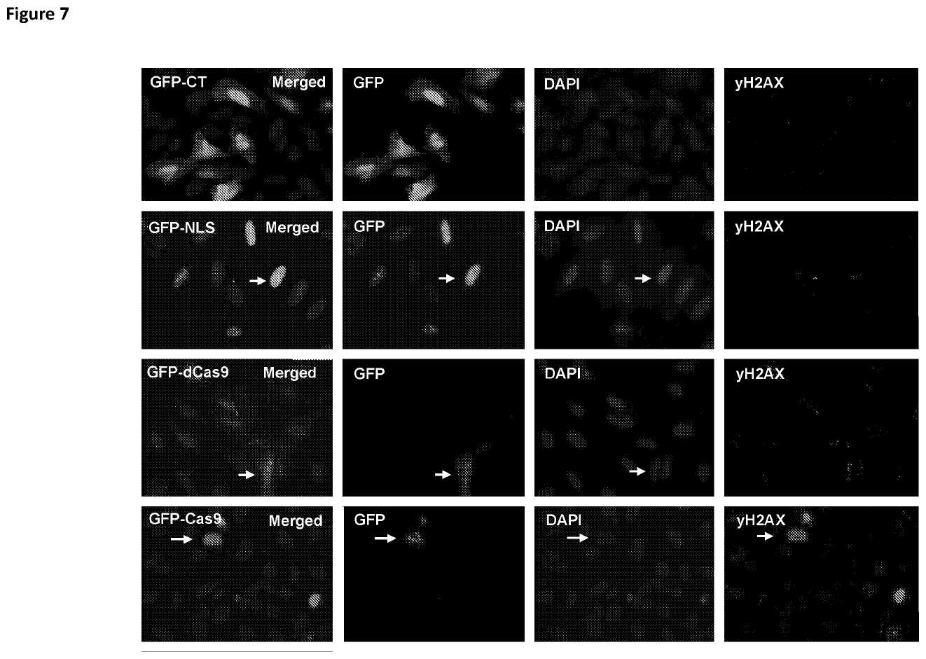

For Campylobacter jejuni, Cas9, dCas9 (inactive) and the nuclear localization signal of Campylobacter jejuni Cas9 were cloned into the cloning site of pEGFP-C1 in frame with GFP. U2OS cells were grown to 40- 50% confluence and then transfected with the plasmids according to the protocol of HP X-tremegene transfection agent (Roche Applied Science, Almere, The Netherlands). After 24 and 48 hours the U2OS cells were washed three times with room temperature HBSS and fixated with 4% paraformaldehyde (Sigma-Aldrich, Zwijndrecht, The Netherlands) and then permeabilized for 20 minutes with 0.1% HBSS—Triton X-100 solution (Sigma-Aldrich, Zwijndrecht, The Netherlands) and background antibody binding was blocked with block buffer (1% fetal bovine serum, 1% Tween-20 (Sigma-Aldrich, Zwijndrecht, The Netherlands), HBSS). Slides were then incubated for one hour with the respective primary antibody for y-H2AX at a 1:100 dilution in block buffer. The appropriate secondary antibodies from the IgG class (H+L), A594 labeled (Molecular Probes, Bleiswijk, The Netherlands), providing a red stain was used to detect y-H2AX thereby revealing that Campylobacter jejuni GFP-Cas9 or the GFP-NLS of Cas9 transfected to the eukaryotic U2OS allowed the localization of GFP to the nucleus cells and more importantly Cas9 induced severe DNA damage as visualized by the y-H2AX staining in cells transfected with active Cas9, but not in the inactive or control cells, see white arrow. These results established that the induced DNA damage is strongly dependent on Cas9 only and that the ability to do so is not dependent on the presence of any added guide RNA (FIG. 7).

### Example 9

### DNA Repair is Active in U2OS Cells when CjCas9 is Absent

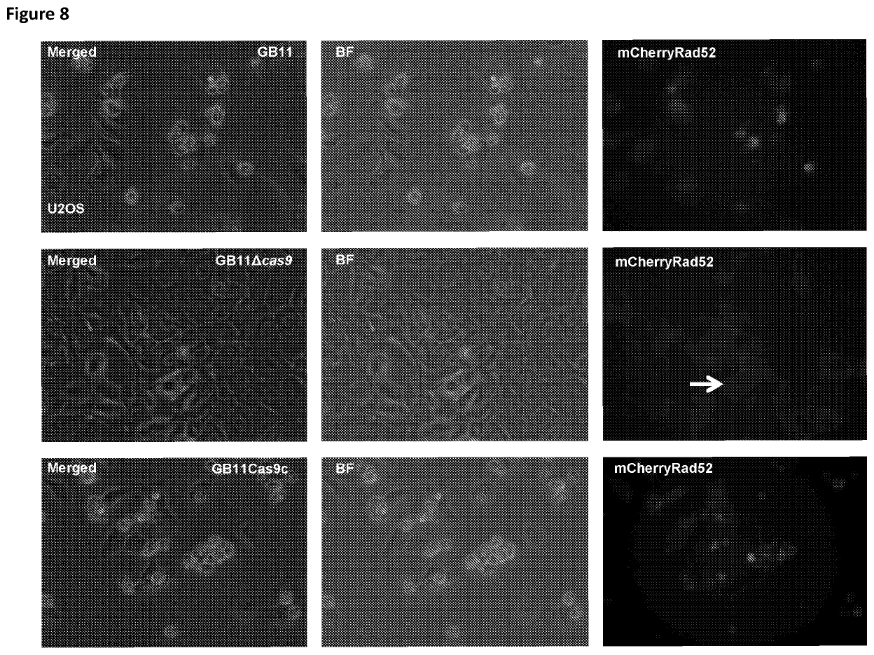

Our pEF1a-mCherry-Rad52 plasmid was kindly provided by Prof. Roland Kanaar (Essers et al., 2002). The plasmid was transfected to U2OS cells using the HP X-tremegene transfection agent (Roche Applied Science, Almere, The Netherlands). Stable, clonal cell lines were established by neomycin selection. U2OS cells positive for Rad52mCherry were grown to 40% to 50% confluence on a 12 well plate (Greiner Bio-one), and Campylobacter jejuni wild type, Δcas9 and Δcas9Δ were inoculated at an MOI of 100 and the infection process was followed over time microscopically using the IX51 phase contrast microscope (Olympus, Leiderdorp, The Netherlands). After 48 hours, cells that were infected with Campylobacter jejuni positive for Cas9 became apoptotic whereas the absence of Cas9 was accompanied with the presence of healthy cells and showed ongoing DNA repair in the nucleus visualized by Rad52mCherry, see white arrow (FIG. 8).

### Example 10

### BLESS Analysis Reveals the induction of Cas9 Dependent Double Strand DNA Breaks

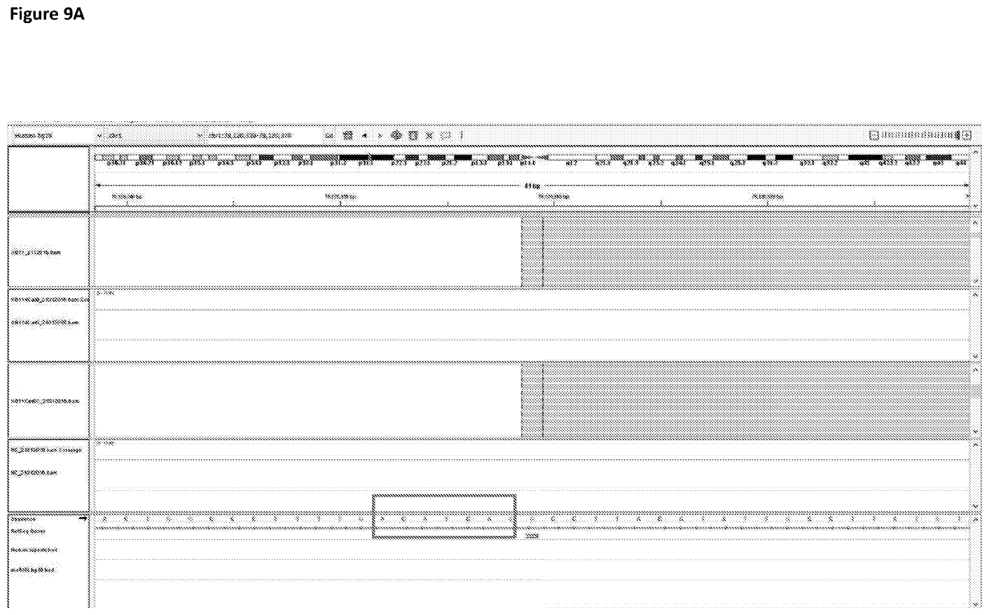

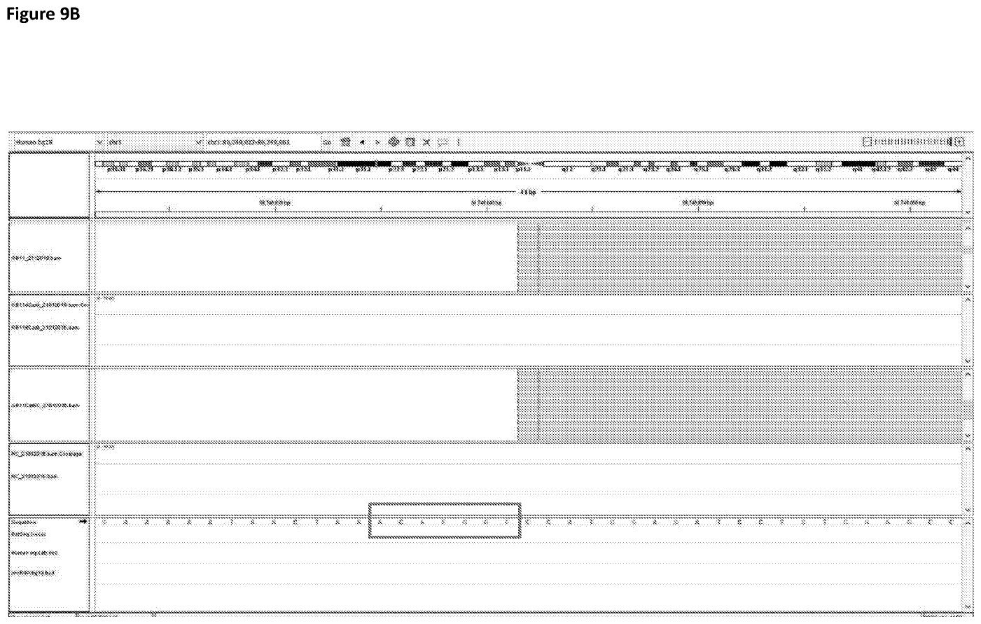

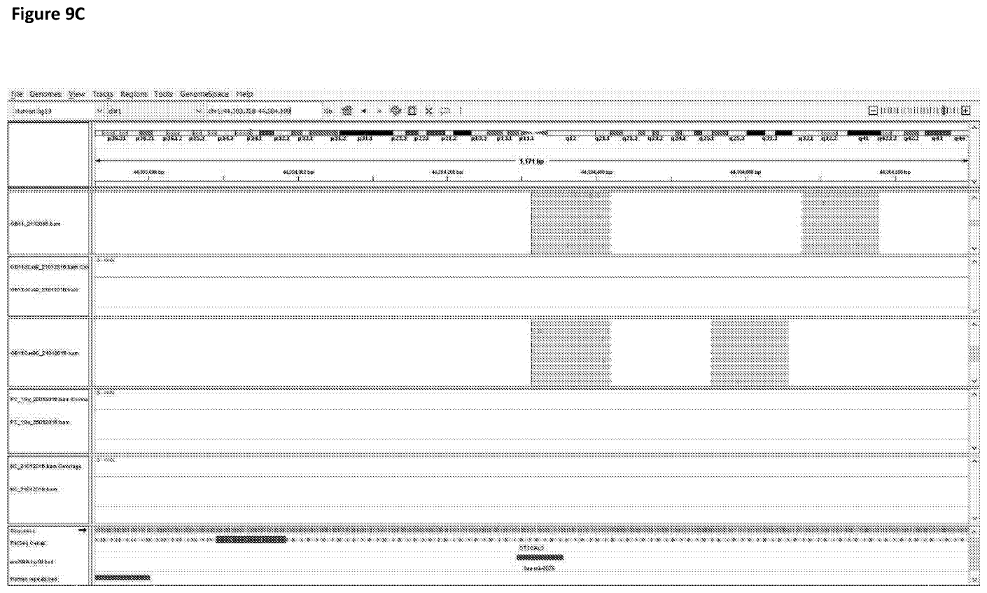

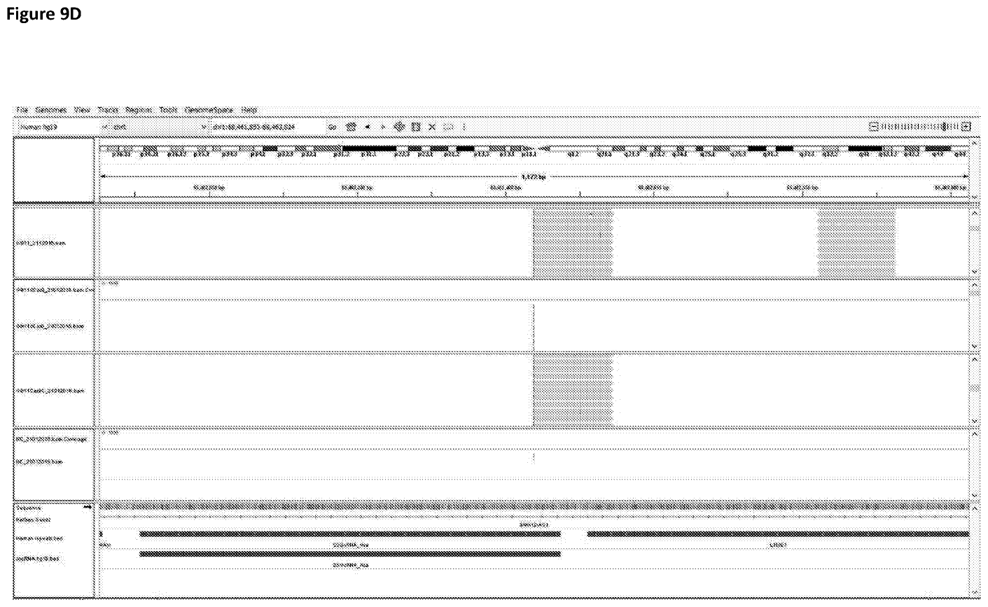

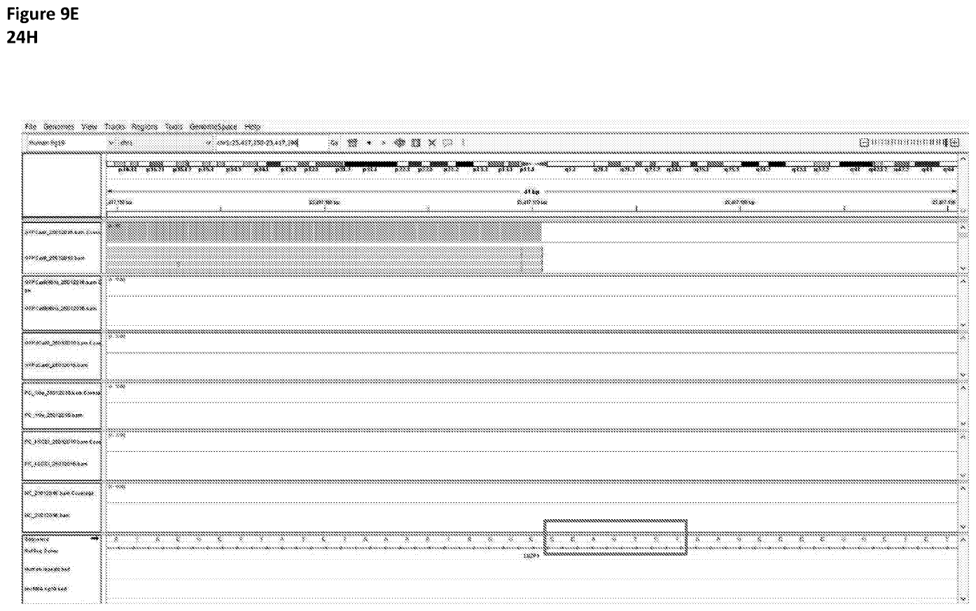

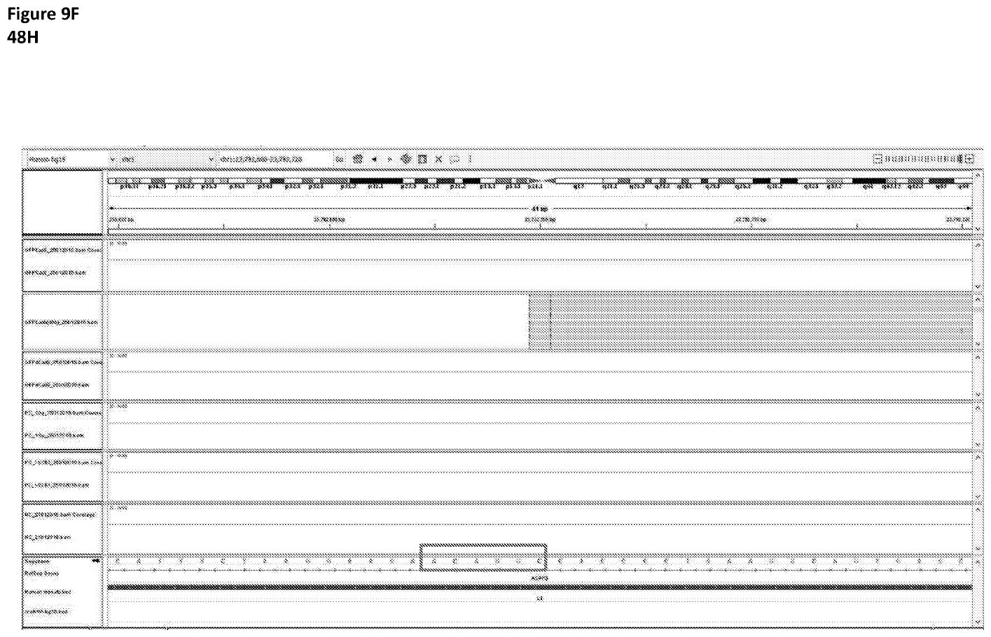

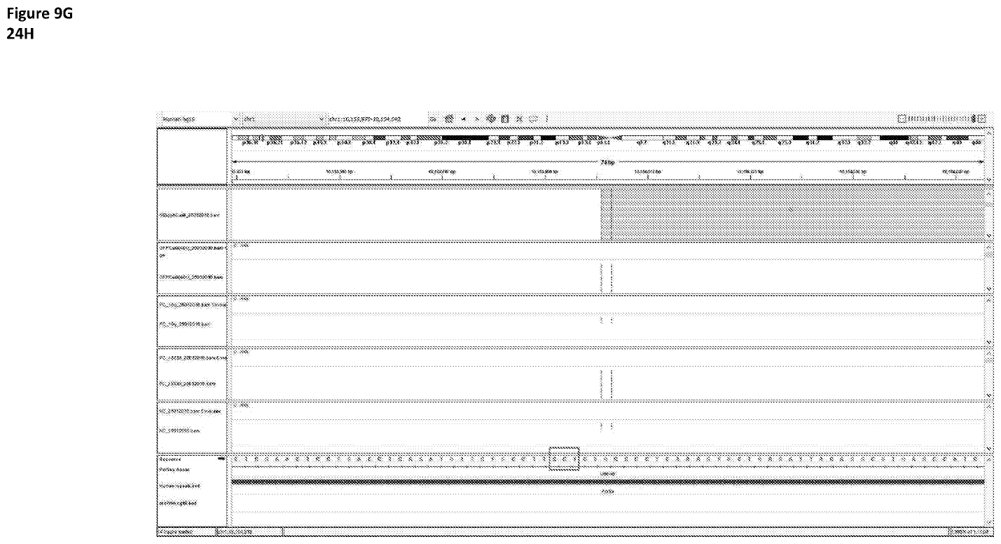

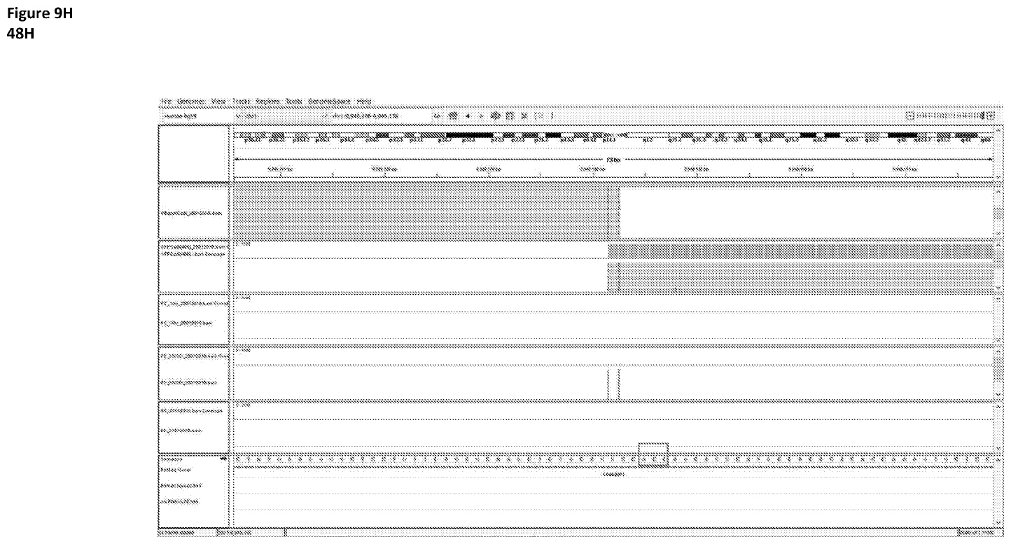

For the BLESS analysis the detailed protocol of Crosetto et al. Nature methods April 2013 was used. U2OS cells were infected for 6 hours with Campylobacter jejuni wild type, Δcas9 and Δcas9Δ and then fixated according to the Crossetto protocol and further processed for PCR and sequencing. Or, U2OS cells were transfected with pEGFP+Cas9, pEGFP+dCas9, pCDNA3.1+Spy-hCas9, pCDNA3.1+CjCas9 using HP X-tremegene transfection agent (Roche Applied Science, Almere, The Netherlands), radiated with 1 Gy or untreated and after 24 or 48 hours fixated according to the Crossetto protocol and further processed for PCR and sequencing. As a positive control U2OS cells with a stable integrated I-SceI site were transfected with a plasmid expressing the I-SceI enzyme harboring a nuclear localization site after 24 hours cells were fixated according to the Crosetto protocol and further processed for PCR and sequencing (the U2OS-I-SceI cell line and plasmid containing the I-Scel-nls enzyme were kindly provided by Prof. Dik van Gent (Erasmus MC). Analysis occurred according the Crosetto protocol with the addition that the obtained sequences were also mapped against non-coding RNAs. In brief, we analyzed Illumina data using the Galaxy software from the bioinformatic department (Erasmus MC). We used a generated pipeline (https://bioinf-galaxian.erasmusmc.nligalaxy/workflow) in which sequences of strand 1 and sequences of strand 2 were uploaded into the galaxian server separately and in order. In short, thereafter the sequences were controlled on quality using FastQC, concatenated and mapped with BWA-MEM against the hg19 genome and analyzed on the number of breaks induced, positions of the breaks, whether they were PAM motif dependent or involved non-coding RNA from repetitive sequences or other known non-coding RNAs. Examples of the obtained data are provided in FIGS. 9A - D and Table 2,). Secondly, some of the Cas9 induced breaks during a Campylobacter jejuni infection of U2OS cells (hotspots) could be confirmed when Cas9 of Campylobacter jejuni was cloned into pEGFP-C1 and transfected to the U2OS cells, but not in the inactive Cas9 of Campylobacter jejuni that was cloned into pEGFP-C1 and transfected to U2OS cells, nor in the 1 Gy radiated U2OS cells or I-SceI restriction site containing U2OS cells or normal U2OS cells (FIG. 9E and 9F and Table 2). The same findings were observed for SpyHCas9 (FIG. 9G and 9H). The BLESS obtained Cas9-specific DSBs were mapped onto the human genome and annotated on the eukaryotic genome using Annovar, revealing which genomic regions were most sensitive to CjCas9 nuclease activity (Table 3). We noticed that the majority of the CjCas9-induced breaks occurred in intronic and intergenic regions during infection (94.1%) and after plasmid transfection (94.7%), respectively (Table 3) Furthermore, the annotation of DSBs revealed that the genomic regions targeted by CjCas9 were mainly located in the SINE and LINE1 repeats (infection; 27,93% or transfection; 16.80%, respectively) (Table 3)

Expression of Cas9 in a Campylobacter jejuni strain that naturally lacks CRISPR-Cas (see Louwen et al EUJCMID 2012)

For expression and purification of Campylobacter jejuni cas9 the gene was supplemented into the pseudogene Cj0046 of Campylobacter jejuni strain 81-176 that naturally lacks a CRISPR-Cas system. Earlier, Cj0046 was shown to be useful for complementation in Campylobacter jejuni. A construct was designed using plasmid pE46 useful for cloning the autologous cas9 promoter and the cas9 gene; the selection marker in these constructs was erythromycin. The vector uses a BsmBI site for cloning. To clone the cas9 gene into the pE46 vector the forward primer BSMSBIPROMFw 5′-GATCCGT CT CACATGCGCTGTGATAAAAGATAACTATCAAG-3′ and the reverse primer BSMBIRev 5′-GTACCGTCTCACATGTTATCATTTTTAAAATCTTCTCTTT GTCTAA-3′ were used to amplify the cas9 gene with its own promoter. PCR products were checked for integrity on ethidium bromide agarose gels and purified with the Zymoclean PCR purification kit (Zymo Research Corp., Leiden, The Netherlands) and PCR products were cloned into PE46. The resulting construct was electroporated at 2.5 kV, 200 Ω, 25 _{I}LEF into Escherichia coli Top10 cells (Invitrogen, Breda, The Netherlands), resuspended in 37° C. pre-warmed SOB medium (Invitrogen, Breda, The Netherlands) and allowed to recover by gentle shaking at 37° C. After recovery, 100 μl was plated onto a LB agar plate containing 250 μg/ml erythromycin (Sigma-Aldrich, Zwijndrecht, The Netherlands) for selection. Colonies were screened by colony PCR for the presence of the cas9 gene, by using the primer pair Cas9FW1 5′-CTTGCCAAGACGCCTTGCAAG-3′, Cas9Rev1 5′-GCGCTATG CAGCGTTCATA AC-3′ covering approximately 450bp at the beginning of the cas9 gene and the primer pair Cas9FW2 5′-AGACGCCGAACTTGAATGTGA-3′, Cas9Rev2 5′-CAGCCTTGCTATGTAGCGAGT-3′ covering approximately 450 bp at the end of the cas9 gene. Positive colonies were grown for plasmid DNA isolation (Thermo Fisher Scientific, Breda, The Netherlands) and subjected to different restriction digestions (BamH1, HinDIII and EcoRI) (New England Biolabs, Alphen aan den Rijn, The Netherlands) to confirm integrity of the constructs.

Electrotransformation of Campylobacter jejuni isolates (see Louwen et al, EuJCMID 2012)

On the day of electro-transformation of Campylobacter jejuni the correct numbers of blood agar plates (with and without antibiotic) were put into a 37° C. incubator, to pre-warm. Campylobacter jejuni 81-176 from the inoculated Columbian blood agar plate (Becton Dickinson, Breda, The Netherlands) was harvested using Lysogeny Broth (LB) supplemented with 1× non-essential amino acids (NEAA) (Invitrogen, Breda, The Netherlands) and a spreader. 81-176 was pelleted by centrifugation for 3 minutes at 14000 rpm and resuspended with 1.5 ml of transformation buffer containing 272 mM sucrose (Sigma-Aldrich, Zwijndrecht, The Netherlands), 15% (v/v) glycerol (Sigma-Aldrich, Zwijndrecht, The Netherlands) and lx NEAA (Invitrogen, Breda, The Netherlands), which was repeated one time. 0.5 ml of transformation buffer was used for re-suspension without 1× NEAA (Invitrogen, Breda, The Netherlands) and was aliquoted in 100 μl samples of the competent cells into 1.5 ml centrifuge tubes. The plasmid DNA (2 pg) containing cas9 with its own promoter was suspended into the 100 μl sample of the 81-176 isolate. The mixture was transferred to an electroporation cuvette and pulsed at 2.5 kV, 200 Ω, 25 _{I}LEF. 1 ml of LB broth supplemented with 1× NEAA (Invitrogen, Breda, The Netherlands) was added to the cuvette and mixed by pipetting to re-suspend. 200 μl of the mix from the cuvette was plated and spread onto the surface of a pre-warmed labeled Columbian blood agar plates (Becton Dickinson, Breda, The Netherlands). Plates were incubated for ˜5 hours at 37° C. under micro-aerobic conditions. The cells were recovered from the plate using 2 ml of LB broth supplemented with 1× NEAA and a spreader. The suspension was spread on the surface of a new pre-warmed blood agar plate supplemented with erythromycin (Sigma-Aldrich, Zwijndrecht, The Netherlands) at a concentration of 0.01 μg/ml to screen for supplemented cas9 positive 81-176 clones. Plates were incubated at 37° C. under micro-aerophilic conditions for 2-5 days, until colonies are visible. Colonies resistant to erythromycin were passaged 5 times on new plates to generate stable clones. From these stable clones genomic DNA was isolated using QlAamp® DNA tissue stool kit (Qiagen, Venlo, The Netherlands) for further analysis. A PCR assay was used to test for correctness of the supplementation assay. For supplementation we used the primer pairs (Fwl) 5′-CATTTGAGTGTTCTAAAAGCTCTTTAGTTT-3′, (Rev1) 5′-GCTTTTACAAGCTC TACTGTTAGTTTGATTG-3′ and (Fw2) 5′-CAAGGCTAAGGATTCTCCTGTT TTGGGATT-3′, (Rev2) 5′-CTTAAGCAAAATCAAATCGTAGCAAC-3′ to confirm that the cas9 supplemented strains had the gene inserted in the sense orientation.

SDS-PAGE and Western Blot (see Louwen et al, EuJCMID 2012)

Fresh overnight cultures of the 11168 strain positive for cas9, 81-176 lacking cas9 or 81-176 supplemented with cas9 were harvested in ice cold PBS and lysed using lysis Matrix B (Sanbio, Uden, The Netherlands) in the Fastprep FP120 machine (Thermosavant) for 25 s at speed 6. Lysates were centrifuged at 14000rpm for 5 minutes at 4° C. and lysated strains were electrophoresed on a 12% SDS-PAGE gel. Gels were blotted onto a PVDF nitrocellulose membrane. Membranes were blocked with PBS containing 5% non-fat dry milk (BioRad, Veenendaal, The Netherlands) and 0.01% Tween-20 (Sigma-Aldrich, Zwijndrecht, The Netherlands) for 1 h at room temperature. Detection of Cas9 was done with the polyclonal antibodies generated in Rabbits against CjCas9 (1:1000) and a secondary antibody anti-rabbit IgG alkaline phosphatase labeled (1:1000). Visualization occurred with NBT/BCIP solution (Sigma-Aldrich, Zwijndrecht, The Netherlands)

Purification of CjCas9 from Campylobacter jejuni

Fresh overnight Campylobacter jejuni cultures of 81-176 +cas9 were harvested in ice cold PBS and lysed using lysis Matrix B (Sanbio, Uden, The Netherlands) in the Fastprep FP120 machine (Thermosavant) for 25 s at speed 6. Lysate was centrifuged at 14000 rpm for 5 minutes at 4° C. To purify CjCas9 the positive charge of Cas9 allowed a cation exchange chromatography step (Pharmacia SP-sepharose) before exposing CjCas9 to affinity purification with monoclonal antibodies bound to sepharose beads targeting Cas9. Additionally, importin alpha one harbors a protein motif that perfectly fits the binding pocket of CjCas9. Labeling of this amino acid sequence in the same structural conformation as present in importin one alpha to sepharose beads allows us by affinity purification to obtain pure Cas9 without any bound bacterial RNA. Purified Cas9 was dialyzed against 10 mM Tris-HCl, 300 mM NaCl and stored in 300 mM NaCl, 10 mM Tris-HCl, 0.1 mM EDTA, 1 mM DTT and 50% Glycerol at pH 7.4 at 25° C.

Cross-linking of Cas9 and a guide RNA

To radioactively label the designed guide of interest occurred according to the HiScribe™ T7 High Yield RNA Synthesis Kit (New England Biolabs, Alphen aan den Rijn, The Netherlands). The 32P-labeled guide RNA was purified with a SPIN-Pure column (G-50). In brief, the column was allowed to adapt to room temperature for a minimum of 30 min before use. Then the column was gently inverted for several times to suspend the column buffer (DEPC-Water), where after the column buffer was allowed to drain by gravity force before proceeding. The column/tube apparatus was placed into an adaptor tube and centrifuged at 1,100×g for 2 min at room temperature. Centrifugation was repeated again to let the column dry completely, where after the collection tube and the eluted buffer were discarded. The column was put in a second collection tube in upright position and the radioactive guide RNA sample (20 to 50 μl) was applied to the center of the column gel in a slowly and gently manner (the rest RNA sample can be stored at −80° C. for one week). Then the column was centrifuged again at 1,100×g for 4 minutes. The purified 32P-labled guide RNA was collected in the bottom of the collection tube and the spin column was discarded and the counts per minute (cpm) value was counted. To bind the guide RNA to Cas9 the following mixture was prepared on ice: 32P-labled guide RNA (2×105 cpm), 1 μl 12.5 mM ATP, 3 μl 5× binding buffer, 3 μl 0.5 M KCl and at least 100 ng of Cas9 by using H2O to final volume was set at 15 μl. The Cas9 and guide RNA were mixed by pipetting up and down for 10 times and then for cross-linking placed with the uncovered tube containing the reaction mixture on ice, directly underneath the bulb (about 10 cm from the surface) of a 254-nm UV light source set to irradiate with 4×105 μJ/cm2 energy. CjCas9 or commercial available SpyHCas9 (New England Biolabs) were UV-cross-linked to the radioactive guide RNA, incubated at room temperature for an additional 30 minutes and kept on ice for another three hours. To remove the non-incorporated 32P, 1 μl of RNase T1 (1 U/μl) was added to each tube and incubate for additional 10 min at 37° C. to degrade the unbound guide RNA. By adding an equal volume (16 μl) of 2× SDS loading dye and separating the sample in a SDS-PAGE gel (no boiling need) a protein shift was visualized by autoradiography.

EGFP gene correction in a K562 cell line.

K562 and K562GFPmut cells were kindly provided by Mali et al. Both cell lines were maintained in DMEM medium (Life technology, Bleiswijk, The Netherlands) supplemented with 10% Foetal Bovine Serum (Life Technology, Breda, The Netherlands) and 1× Non-Essential Amino Acids (Life Technology, Breda, The Netherlands). The cells were routinely grown in a 75-cm2 flask (Greiner Bio-One, Alphen aan den Rijn, The Netherlands) at 37° C. in a humidified 5% CO2-95% air incubator (Binder, Tuttlingen, Germany). From confluent stock cultures an aliquot at a concentration of 5.0×105 cells/ml was seeded into a new flask and fresh medium. To restore the GFP expression and to control the laboratorial generated Cas9xguideRNA complex of CjCas9 or SpyHCas9 and/or GFP template were microinjected in combination or separately according to the protocol of Bao et al, Scientific Reports (2015) into the K562 and K562GFPmut cells. Single cells were allowed to form clones for 14-16 days. Genomic DNA was isolated using the PureLink® Genomic DNA Mini Kit (Thermofisher scientific, Breda, The Netherlands). Off targeting was analyzed by whole genome sequencing of the K562, K562GFPmut cells and the generated clones after Cas9xguideRNA complex and/or GFP template microinjection at Service XS. Using bioinformatics we compared the genomes and analyzed for SNP's, Indel's and a-specific recombination of the GFP template into the eukaryotic genome of the K562 or K562GFPmut cells.

### Example 11

### Bacterial SpyCas9 effectuates genome editing

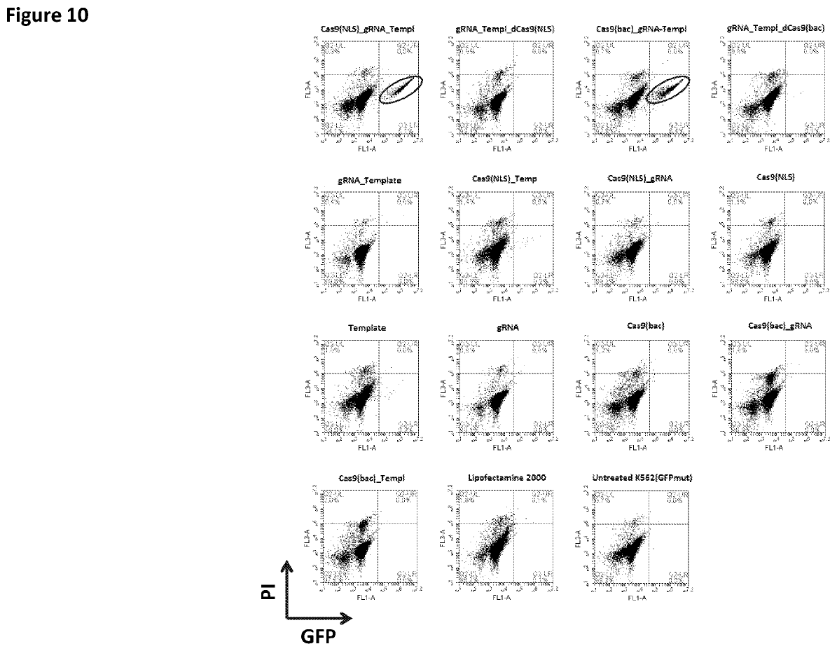

For genome editing and FACS analyses we used the K562(GFPmut) cell line*, which was seeded into a 24-well plate (Greiner Bio-one, Alphen aan den Rijn, The Netherlands). After overnight recovery we transfected the K562(GFPmut) cells using Lipofectamine 2000 (Thermofisher scientific, Breda, The Netherlands), using an RNP complex of the human optimized SpyHCas9 (New England Biolabs, Massachusetts, United States) or the bacterial SpyCas9 (New England Biolabs, Massachusetts, United States) reversibly complexed with a synthetic guide RNA (Biolegio, Nijmegen, The Netherlands) and a PCR product generated from the GFP gene. To form the active RNP complex the protocol obtained from the manufacturer was used. Transfection using Lipofectamine 2000 (Thermofisher scientific, Breda, The Netherlands) occurred according to the manufacturer's protocol. The synthetic guide RNA exactly matched the guide RNA GFP2 sequence as described previously *. Seventy two hours after transfection the K562(GFPmut) cells were harvested using ice cold HBSS (Thermofisher scientific, Breda, The Netherlands) and kept on ice; after the second wash the cells were resuspended in 0.4 ml HBSS (Thermofisher scientific, Breda, The Netherlands) containing propidium iodide (Sigma-Aldrich, Zwijndrecht, The Netherlands) (2μl/10 ml HBSS solution). The K562(GFPmut) cells were measured on a FACS Calibur or FACS Canto II HTS and data was analysed using FlowJo software (version 8.8.6). FL3-A shows the number of alive or dead cells detected by propidium iodide. FL1-A shows the number of normal or GFP positive cells. For the human optimized SpyCas9(NLS) the number of GFP positive cells was 5.1%, when an equal concentration (400 pmol) of the bacterial SpyCas9 was used the number of GFP positive cells was 6,4%. The example reveals that both the bacterial SpyCas9, visualized as Cas9(bac) and the human optimized SpyCas9 as visualized as Cas9(NLS) can edit human cells and restore the GFP expression as previously described for the human optimized SpyCas9 * (FIG. 10). This indicates that also the bacterial SpyCas9 can enter the eukaryotic nucleus on its own, without any addition of a nuclear localisation signal allowing editing as efficient as obtained for the human optimized SpyCas9. The appropriate controls, the heat inactivated Cas9 of both the bacterial and human optimized SpyCas9, Cas9 proteins alone, gRNA alone, GFP (PCR product) template alone, or combinations that do not result in editing, did not reveal GFP positive cells above background levels.

*Mali, P. et al. RNA-guided human genome engineering via Cas9. Science (80-.). 339, 823-826 (2013).

### Example 12

### Microscopic Visualization of Bacterial SpyCas9 Mediated Genome Editing

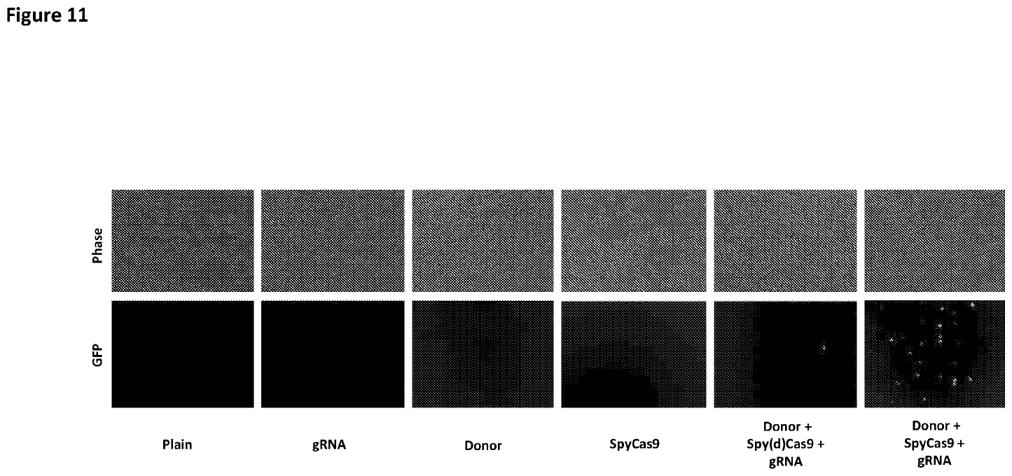

Bright field and GFP fluorescent example pictures as an illustration presented for example 10 were obtained using the IX51 microscope (Olympus, Leiderdorp, The Netherlands). The pictures reveal. The bacterial or human optimized SpyCas9 were allowed to form a reversible complex with the synthetic gRNA using the protocol as obtained from the manufacturer Biolegio. The RNP complex is transfected together with the GFP (PCR product) template using Lipofectamine 2000 (Thermofisher scientific, Breda, The Netherlands) onto K562(GFPmut) cell line. The pictures show that only with the Cas9/gRNA RNP complex and GFP template a significant amount of GFP positive cells is detected (FIG. 11). The GFP positive cells are absent in the controls.

### Example 13

### gRNA Enrichment Reduces SpyCas9 Toxicity (Apoptosis)

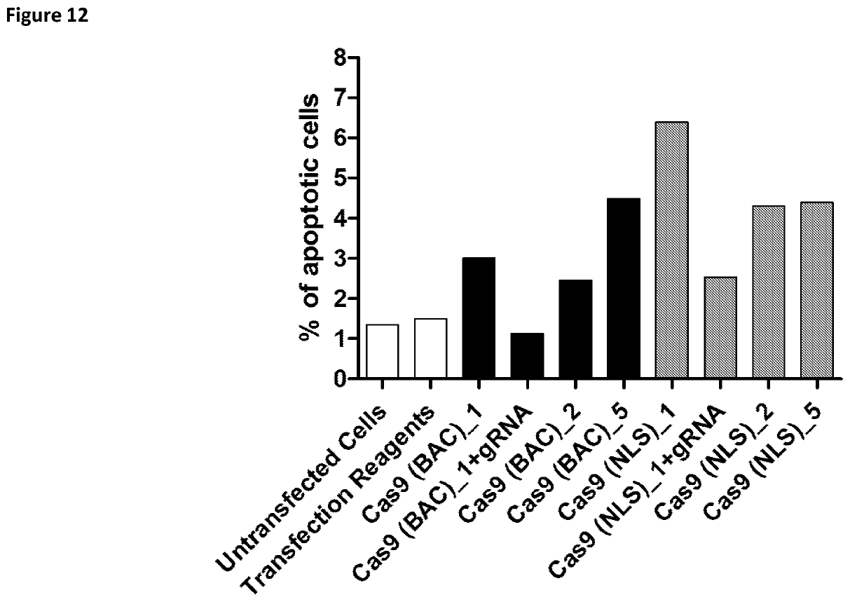

The experiment as described in example 10 was repeated, but limited to the parent cell line K562 * and the following combination (untransfected K562 cells, K562 cells transfected with lipofectamine 2000 only, K562 cells transfected with Cas9(Bac)_1(80 pmol), Cas9(Bac)_1(80 pmol) and synthetic gRNA GFP2 *(80 pmol), Cas9(Bac)_2(200 pmol), Cas9(Bac)_5(500 pmol), Cas9(NLS)_1(80 pmol), Cas9(NLS)_1(80 pmol) and synthetic gRNA GFP2 *(80 pmol), Cas9(NLS)_2(200 pmol), Cas9(NLS)_5(500 pmol). After overnight incubation the K562 cells were harvested and analysed on apoptosis induction using an Annexin V/Propidium Iodide Apoptosis Assay (Thermofisher scientific, Breda, The Netherlands) for accurate assessment of cell death *. Y-axis reveals the percentage of apoptotic K562 cells detected by FACS analyses. X-axis shows the combinations of Cas9, gRNA and additional controls that were tested. This experiment reveals that for both the bacterial SpyCas9(Bac) and the human optimized SpyCas9(NLS), gRNA saturation is required to reduce the toxic effects of Cas9 alone (FIG. 12).

*Rieger et al Modified Annexin V/Propidium Iodide Apoptosis Assay For Accurate Assessment of Cell Death. J Vis Exp. (50): 2597 (2011).

### Example 14

### gRNA Enrichment Reduces the Number of CjCas9 Induced Double Stranded Breaks

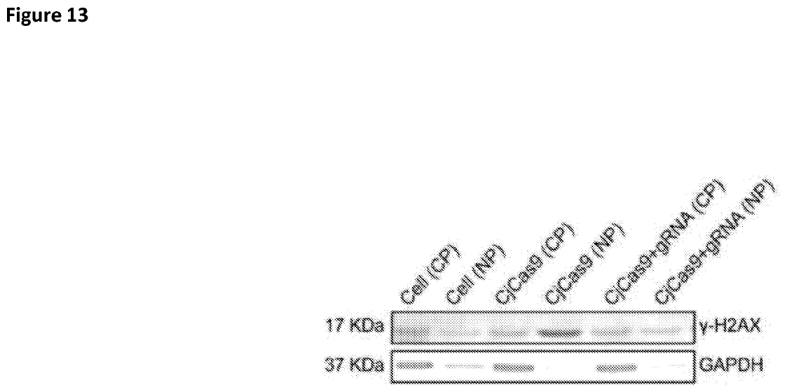

In the construct pCDNA3.1 Cas9 of Campylobacter jejuni or the gRNA GFP2 * is cloned. The constructs were transfected as follows (pCDNA3.1 +CjCas9 alone) or (pCDNA3.1+CjCas9 and pCDNA3.1+gRNA GFP2) by using the HP X-tremegene transfection agent (Roche Applied Science, Almere, The Netherlands) and HEK293T cell line that were grown to 90% confluence in a 12 well cell culture plate (Greiner Bio-one, Alphen aan den Rijn, The Netherlands). For cytoplasmic and nuclear protein fractions NE-PER™ Nuclear and Cytoplasmic Extraction Reagents (TFS) were used and proteins were extracted according to the manufacturer's instructions (Thermofisher scientific, Breda, The Netherlands) after 24 hours. For detection of DSBs, a SDS-PAGE (4-20%) gradient gel (Biorad, Veenendaal, The Netherlands) was run and Western blotted. The membranes were immune-probed using a primary mouse monoclonal anti-phospho-Histone H2A.X (Ser139) clone JBW301 (Millipore, Amsterdam, The Netherlands) antibody or mouse-anti-GAPDH antibody (Abeam, Cambridge, United Kingdom) at a 1:1000 dilution and incubated for 1.5-2 hours at room temperature. Anti-mouse IgG labelled with alkaline phosphatase (Sigma-Aldrich, Zwijndrecht, The Netherlands) at a dilution of 1:1000 was used as a secondary antibody. Targeted proteins bands were visualize using NBT/BCIP solution (Sigma-Aldrich, Zwijndrecht, The Netherlands) according to the manufacturer's instructions. The Western blot reveals that when CjCas9 is saturated by overexpressing a decoy gRNA a strong reduction in DSBs is detected as visualized by the y-H2AX staining The GAPDH loading control reveals that the protocol to separate the cytoplasmic from the nuclear fraction was successful (FIG. 13).

*Mali, P. et al. RNA-guided human genome engineering via Cas9. Science (80-.). 339, 823-826 (2013).

### Example 15

### gRNA is Bound to SpyCas9 after UV-Crosslinking

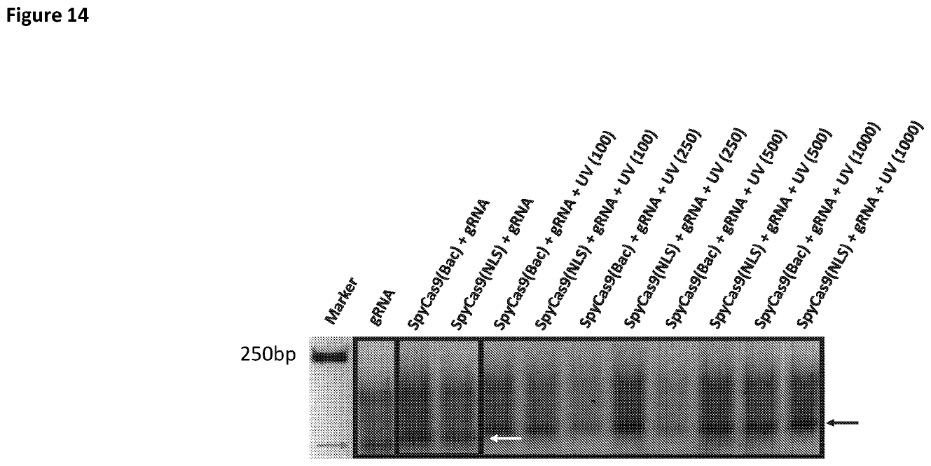

In a microfuge tube different combinations of the synthetically produced gRNA GFP2 (20 pmol), tracrRNA oligo (100 pmol), bacterial SpyCas9(Bac) (New England Biolabs, Mass., United States) 20 pmol, human optimized SpyCas9(NLS) (New England Biolabs, Mass., United States) 20 pmol and the GFP template PCR product were generated in nuclease free water and a 10× SpyCas9 buffer both obtained from (Biolegio, Nijmegen, The Netherlands) in a total volume of 20 μl. The SpyCas9(Bac) or SpyCas9(NLS) in (reversible) complex with the synthetic guide RNA were or were not exposed to UV radiation (254nm) using the CL-1000 Ultraviolet crosslinker (VWR, Amsterdam, The Netherlands) and 100, 250, 500, 1000×100 μJ/cm³ exposure variations. Then loading buffer was added and the samples were run using an agarose gel electrophoresis protocol with 3% agarose (Sigma-Aldrich, Zwijndrecht, The Netherlands), Tris-Borate EDTA buffer (Thermofisher Scientific, Breda, The Netherlands) and SYBRsafe (Thermofisher Scientific, Breda, The Netherlands) at 50mA for 2,5 hours. Hereafter the guide RNA was visualized using a gel doc system (Biorad, Massachusetts, United States) and a picture was taken, showing that the gRNA runs at a different position when in combination with SpyCas9(Bac) or SpyCas9(NLS); or after exposure to different UV radiation intensities, showing that UV cross-linking resulted in a DNA electrophoretic mobility shift (FIG. 14).

### Example 16

### Irreversible Bound RNP Complex of SpyCas9(NLS) and gRNA Allows Genome Editing

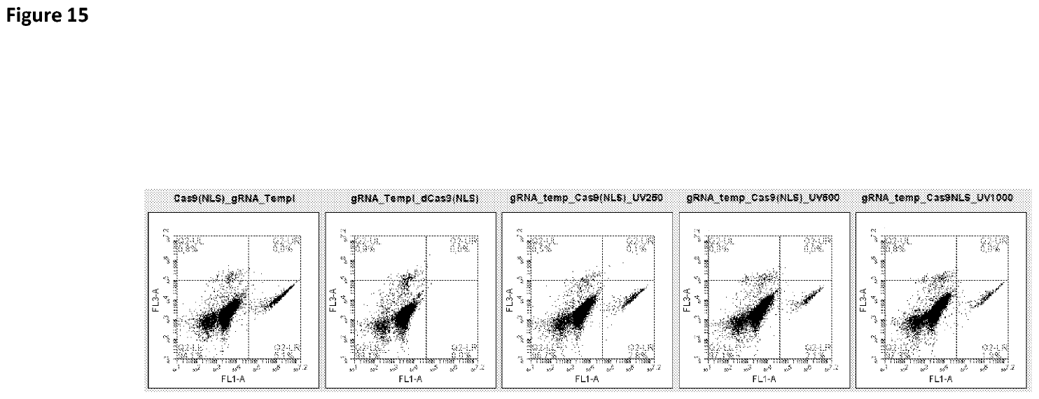

For genome editing and FACS analyses we used the K562(GFPmut) cell line*, which was seeded into a 24-well plate (Greiner Bio-one, Alphen aan den Rijn, The Netherlands). After overnight recovery we transfected the K562(GFPmut) cells using Lipofectamine 2000 (Thermofisher scientific, Breda, The Netherlands), using an RNP complex of the human optimized SpyCas9 NLS (New England Biolabs, Mass., United States) in its inactive variant or the RNP complex exposed to different intensities of UV radiation 250, 500, 1000 ×100μJ/cm³. Seventy two hours after transfection the K562(GFPmut) cells were harvested using ice cold HBSS (Thermofisher scientific, Breda, The Netherlands) and kept on ice; after the second wash the cells were resuspended in 0.4 ml HBSS (Thermofisher scientific, Breda, The Netherlands) containing propidium iodide (Sigma-Aldrich, Zwijndrecht, The Netherlands) (2 μl/10 ml HBSS solution). The K562(GFPmut) cells were measured on a FACS Calibur or FACS Canto II HTS and data was analysed using FlowJo software (version 8.8.6). FL3-A shows the number of alive (Q2-LL) or dead cells (Q2-UL) detected by propidium iodide. FL1-A shows the number of normal (Q2-LL) or GFP positive cells (Q2-LR). For the human optimized SpyCas9(NLS) the number of GFP positive cells was 5.1%, for the RNP complex UV crosslinked with 250, 500, 1000×100 μJ/cm³ intensities, the editing efficiency as measured by GFP positive cells was 2,8; 2,1 or 1,9% (FIG. 15), showing that after UV exposure the RNP complex (SpyCas9(NLS)/gRNA) still allows genome editing.

TABLE 1
Primer list and their corresponding restriction site plus BLESS linkers
Restriction site specific primers

| | | Restriction | |
|---|---|---|---|
| Primer name | Direction | site | Primer sequence (5′-3′) |
| pEGFP-NFw | Forward | Nhe I | ATCCGCTAGCATGGCAAGAATTTTGGCATTTGATAT |
| | | | A |
| pEGFP-NRev | Reverse | Xho I | TGAGCTCGAGTTTTTTAAAATCTTCTCTTTGTCTAAA |
| pEGFP-CFw | Forward | Sal I | TGCAGTCGACATGGCAAGAATTTTGGCATTTGATAT |
| | | | A |
| pEGFP-CRev | Reverse | Bam HI | CGGTGGATCCTTTTTTAAAATCTTCTCTTTGTCTAAA |
| pEGFPC1_CT(For) | Forward | Sal I | TGCAGTCGACAGCAACAGTGCAGAACTTTATGCA |
| pEGFPC1_CT(Rev) | Reverse | Bam HI | CGGTGGATCCTTGCCTAAAACCACTAAAAGGCAC |
| pEGFPC1_NT(For) | Forward | Sal I | TGCAGTCGACGGAGAATCCTTAGCCTTGCCA |
| pEGFPC1_NT(Rev) | Reverse | Bam HI | CGGTGGATCCTAAATGTTTTAAGTGATTTAG |
| pEGFPN1_CT(For) | Forward | Nhe I | ATCCGCTAGCATGAGCAACAGTGCAGAACTTTATGC |
| | | | A |
| pEGFPN1_CT(Rev) | Reverse | Xho I | TGAGCTCGAGTTGCCTAAAACCACTAAAAGGCAC |
| pEGFPN1_NT(For) | Forward | Nhe I | ATCCGCTAGCATGGGAGAATCCTTAGCCTTGCCA |
| pEGFPN1_NT(Rev) | Reverse | Xho I | TGAGCTCGAGTAAATGTTTTAAGTGATTTAG |
| SpyCas9_pEGFP-C1 | Forward | SalI | TGCAGTCGACATGGATAAGAAATACTCAATAGGCTT |
| | | | A |
| SpyCas9_pEGFP-C1 | Reverse | Bam HI | CGGTGGATCCGTCACCTCCTAGCTGACTCAAATCAA |
| | | | T |
| NmCas9_pEGFP-C1 | Forward | SalI | TGCAGTCGACATGGCTGCCTTCAAACCTAATTCAAT |
| NmCas9_pEGFP-C1 | Reverse | Bam HI | CGGTGGATCCACGGACAGGCGGGCGTTTTTTCAGA |
| | | | CG |
| SpyCas9_pEGFP-C1 | Reverse | SacII | GGGCCCGCGGGTCACCTCCTAGCTGACT |
| pSpyCas9GE | Forward | AflII | AACTTAAGATGGATAAGAAATACTCAATAGG |
| pSpyCas9GE | Reverse | XhoI | GACTCGAGTCAGTCACCTCCTAGCTG |
| pNmCas9GE | Forward | NheI | TGGCTAGCATGGCTGCCTTCAAACCTA |
| pNmCas9GE | Reverse | XhoI | GACTCGAGTTAACGGACAGGCGGGC |
| NLS(SpyCas9) | Forward | SalI | TGCAGTCGACGCGGAAGCGACTCGTCTCAA |
| NLS(spyCas9) | Reverse | Ban HI | CGGTGGATCCCTCCTGTAGATAACAAATACG |
| NLS(FnCas9) | Forward | SalI | TGCAGTCGACTTGATGAATAATAGAACAGCA |
| NLS(FnCas9) | Reverse | Bam HI | CGGTGGATCCCTTAAAGAGCCTTTTGACTAG |
| NLS(NmCas9) | Forward | SalI | TGCAGTCGACGCCATGGCAAGGCGTTTGGCG |
| NLS(NmCas9) | Reverse | Bam HI | CGGTGGATCCTAGGCGGCGGGT |
| BLESS P1 | | | CCCTAGCGTAACTCTCGAGGTAGTA |
| BLESS P2 | | | CCCTAGCGTAACTAGGCCACTCGAG |
| BLESS P3 | | | CTAGCGTAACTCTCGAGACGACG |

| | |
|---|---|
| BLESS P4 | GTATCGCCTCCCTCGCGCCATCAGACGAGTGCGTCCCTAGCGTAACTCTCGAG |
| | GTAGTA |
| BLESS P5 | CTATGCGCCTTGCCAGCCCGCTCAGACGAGTGCGTCTAGCGTAACTCTCGAGA |
| | CGACG |
| Linker 1 PL1 | TACTACCTCGAGAGTTACGCTAGGGATAACAGGGTAATATAGTTT(T)- |
| | biotinTTTCTATATTACCCTGTTATCCCTAGCGTAACTCTCGAGGTAGTA |
| Linker 2 DL2 | CTCGAGTGGCCTAGTTACGCTAGGGATAACAGGGTAATATAGTTTTTTTCTATA |
| | TTACCCTGTTATCCCTAGCGTAACTAGGCCACTCGAG |
| Linker 3 DL3 | CGTCGTCTCGAGAGTTACGCTAGGGATAACAGGGTAATATAGTTTTTTTCTATA |
| | TTACCCTGTTATCCCTAGCGTAACTCTCGAGACGACG |

TABLE 2
Total numbers of DSBs detected with the BLESS analysis.

| | Total | Filter | Only in | Not in | |
|---|---|---|---|---|---|
| | break | (>3 | Chr. | Refer- | |
| Sample | sites | reads) | 1: 22X | ence* | Overlap |
| C. jejuni | 1633494 | 690299 | 686206 | 678910 | |
| GB11 (WT) | | | | | |
| C. jejuni | 727672 | 191271 | 190111 | | |
| GB11Δcas9 | | | | | |
| C. jejuni | 2150945 | 956998 | 951632 | 942104 | 25539** |
| GB11Δcas9::cas9 | | | | | |
| pEGFP-CjCas9 | 722654 | 203758 | 202496 | 199326 | 833*** |
| (24 h) | | | | | |
| pEGFP-CjCas9 | 824219 | 260425 | 258885 | 255186 | 1189**** |
| (48 h) | | | | | |
| pEGFP-CjdCas9 | 654496 | 166571 | 165817 | | |
| (24 h) | | | | | |
| pCjCas9 (24 h) | 795106 | 236446 | 235374 | 231912 | 1102**** |
| 1Gy | 554956 | 157006 | 153590 | | |
| I-SceI | 800235 | 277093 | 271970 | | |
| U2OS (cell only) | 34762 | 94039 | 93373 | | |

*Reference: Bacterial samples = C. jejuni GB11Acas9 + U2OS (cell only); Plasmid samples = pEGFP-CjdCas9 (24 h) + U2OS (cell only) or in case for pCjCas9, U2OS cell only.
**Common DSBs sites between C. jejuni GB11 and GB11Δcas3::cas3 after removing reference.
***Common DSBs sites between common bacterial DSBs (25539) and pEGFP-CjCas9 (24 h) plasmid sample after removing reference.
****Common DSBs sites between common bacterial DSBs (25539) and pEGFP-CjCas9 (48 h) plasmid sample after removing reference.
*****Common DSBs sites between common bacterial DSBs (25539) and pCjCas9 (24 h) plasmid sample after removing reference.

TABLE 3
ANNOVAR and repeat annotation of CjCas9 DSBs positions.

| | |
|---|---|
| DSBs site | Percentage (%) |

ANNOVAR annotation of common bacterial DSBs

| | | |
|---|---|---|
| Exonic | 257 | 1.01 |
| Splicing | 1 | 0.00 |
| ncRNA_Exonic | 81 | 0.32 |
| ncRNA_Intronic | 1051 | 4.12 |
| UTR5 | 42 | 0.16 |
| UTR3 | 243 | 0.95 |
| Intronic | 9280 | 36.34 |
| Upstream | 175 | 0.69 |
| Downstream | 159 | 0.63 |
| Intergenic | 14249 | 55.79 |
| (blank) | 1 | 0.00 |
| Total | 25539 | |

ANNOVAR annotation of common (bacterial and plasmid) DSBs

| | | |
|---|---|---|
| Exonic | 6 | 0.54 |
| Splicing | 0 | 0.00 |
| ncRNA_Exonic | 0 | 0.00 |
| ncRNA_Intronic | 30 | 2.72 |
| UTR5 | 0 | 0.00 |
| UTR3 | 9 | 0.82 |
| Intronic | 298 | 27.04 |
| Upstream | 5 | 0.45 |
| Downstream | 7 | 0.64 |
| Intergenic | 746 | 67.70 |
| (blank) | 1 | 0.09 |
| Total | 1102 | |

Repeat annotation of common bacterial DSBs

| | | |
|---|---|---|
| SINE/Alu | 2768 | 10.84 |
| SINE/MIR | 771 | 3.02 |
| LINE/L1 | 2303 | 9.02 |
| LINE/L2 | 899 | 3.52 |
| LINE/CR1 | 101 | 0.40 |
| LINE/RTE-X | 33 | 0.13 |
| LTR/ERVL-MaLR | 1131 | 4.43 |
| LTR/ERV1 | 534 | 2.09 |
| LTR/ERVL | 501 | 1.36 |
| LTR/ERVK | 49 | 0.19 |
| LTR/Gypsy | 36 | 0.14 |
| DNA/hAT-Charlie | 537 | 2.10 |
| DNA/TcMar-Tigger | 299 | 1.17 |
| DNA/hAT-Tip100 | 112 | 0.44 |
| Satellite | 1341 | 5.25 |
| Simple repeat | 46 | 0.18 |
| Other repeat | 284 | 1.71 |
| Unknown | 6 | 0.02 |
| (blank) | 13788 | 53.99 |
| Total | 25539 | |

Repeat annotation of common (bacterial and plasmid) DSBs

| | | |
|---|---|---|
| SINE/Alu | 67 | 6.09 |
| SINE/MIR | 26 | 2.36 |
| LINE/L1 | 64 | 5.72 |
| LINE/L2 | 22 | 2.00 |
| LINE/CR1 | 6 | 0.54 |
| LINE/RTE-X | 1 | 0.09 |
| LTR/ERVL-MaLR | 32 | 2.91 |
| LTR/ERV1 | 14 | 1.27 |
| LTR/ERVL | 15 | 1.36 |
| DNA/hAT-Charlie | 18 | 1.63 |
| DNA/TcMar-Tigger | 12 | 1.09 |
| DNA/hAT-Tip100 | 3 | 0.27 |
| Satellite | 193 | 17.52 |
| Simple repeat | 4 | 0.36 |
| Other repeat | 11 | 1.11 |
| (blank) | 613 | 55.68 |
| Total | 1101 | |

1. A Type II Cas-based nuclease complex comprising a Cas protein and a guide RNA sequence, wherein the guide RNA sequence is irreversibly crosslinked to the Cas protein.

2. The complex according to claim 1, wherein the guide RNA sequence comprises a CRISPR nucleic acid sequence.

3. The complex according to claim 1, wherein the Cas protein is Cas9.

4. The complex according to claim 1, wherein the guide RNA is not derived from the same organism as the Cas protein.

5. The complex according to claim 1, wherein the Cas protein is derived from Pasteurella multocida, Streptococcus thermophilus, Streptococcus agalactiae, Streptococcus anginosus, Streptococcus bovis, Streptococcus canis, Streptococcus constellatus, Streptococcus dysgalactiae, Streptococcus equi, Streptococcus equinus, Streptococcus gallolyticus, Streptococcus infantarius, Streptococcus iniae, Streptococcus macacae, Streptococcus mitis, Streptococcus ° rails, Streptococcus gordonii, Streptococcus infantarius, Streptococcus macedonicus, Streptococcus parasanguinis, Streptococcus pasteurianus, Streptococcus pseudoporcinus, Streptococcus ratti, Streptococcus salivarius, Streptococcus sanguinis, Streptococcus suis, Streptococcus pyogenes, Streptococcus mutans, Streptococcus vestibularis, Pediococcus acidilactici, Staphylococcus aureaus, Staphylococcus lugdunensis, Staphylococcus pseudintermedius, Staphylococcus simulans, Escherichia coli, Neisseria bacilliformis, Neisseria cinerea, Neisseria flavescens, Neisseria lactamica, Neisseria meningitides, Neisseria wadsworthii, Listeria innocua, Francisella novicida, Campylobacter jejuni, Campylobacter coli, Campylobacter lari, Helicobacter canadensis, Helicobacter cinaedi, Lactobacillus animalis, Lactobacillus farciminis, Lactobacillus buchneri, Lactobacillus casei, Lactobacillus coryniformis, Lactobacilus farciminis, Lactobacillus fermentum, Lactobacillus forum, Lactobacillus gasseri, Lactobacillus hominis, Lactobacillus finers, Lactobacillus jensenii, Lactobacillus johnsonii, Lactobacillus mucosae, Lactobacillus paracasei, Lactobacillus pentosus, Lactobacillus plantarum, Lactobacillus rhamnosus, Lactobacillus ruminis, Lactobacillus salivarius, Lactobacillus sanfranciscensis, Lactobacillus versmoldensis, Legionella pneumophila, Listeria monocytogenes, Acidaminococcus intestine, Acidothermus cellulolyticus, Acidovorax ebreus, Actinobacillus minor, Actinobacillus pleuropneumonias, Actinobacillus succinogenes, Actinobacillus suis, Actinomyces coleocanis, Actinomyces georgiae, Actinomyces naeslundii, Actinomyces turicensis, Acidovorax avenae, Akkermansia muciniphila, Alicycliphilus denitrificans, Alicyclobacillus hesperidum, Aminomonas paucivorans, Anaerococcus tetradius, Anaerophaga thermohalophila, Bacillus cereus, Bacillus smithii, Bacillus thuringiensis, Bacteroides coprophilus, Bacteroides coprosuis, Bacteroides dorei, Bacteroides eggerthii, Bacteroides faecis, Bacteroides fluxus, Bacteroides fragilis, Bacteroides nordii, Bacteroides uniformis, Bacteroides vulgatus, Barnesiella intestinihominis, Bergeyella zoohelcum, Bifidobacterium bifidum, Bifidobacterium dentium, Bifidobacterium longum, Brevibacillus laterosporus, Caenispirillum salinarum, Capnocytophaga gingivalis, Capnocytophaga canimorsus, Capnocytophaga sputigena, Catellicoccus marimammalium, Catenibacterium mitsuokai, Clostridium perfringens, Clostridium spiroforme, Coprococcus catus, Coriobacterium glomerans, Corynebacterium accolens, Corynebacterium diphtheria, Dinoroseobacter shibae, Dorea longicatena, Dolosigranulum pigrum, Elusimicrobium minutum, Enterococcus faecalis, Enterococcus faecium, Enterococcus hirae, Enterococcus itaficus, Eubacterium dolichum, Eubacterium rectale, Eubacterium ventriosum, Eubacterium yurii, Facklamia hominis, Fibrobacter succinogenes, Filifactor alocis, Finegoldia magna, Flavobacterium branchiophilum, Flavobacterium columnare, Flavobacterium psychrophilum, Fluviicola taffensis, Francisella tularensis, Fructobacillus fructosus, Fusobacterium nucleatum, Gardnerella vaginalis, Gemella haemolysans, Gemella morbillorum, Gluconacetobacter diazotrophicus, Gordonibacter pamelaeae, Haemophilus parainfuenzae, Haemophilus sputorum Helcococcus kunzii, Helicobacter mustelae, lndibacter alkaliphilus, lgnavibacterium album, llyobacter polytropus, Joostella marina, Kordia algicida, Leuconostoc gelidum, Methylosinus trichosporium, Mucilaginibacter paludis, Myroides injenensis, Myroides odoratus, Mobiluncus curtisii, Mobiluncus mulieris, Mycoplasma canis, Mycoplasma gallisepticium, Mycoplasma mobile, Mycoplasma ovipneumoniae, Mycoplasma synoviae, Niabella soli, Nitratifractor salsuginis, Nitrobacter hamburgensis, Odoribacter laneus, Oenococcus kitaharae, Ornithobacterium rhinotracheale, Parabacteroides johnsonii, Parasutterella excrementihominis, Parvibaculum lavamentivorans, Phascolarctobacterium succinatutens, Planococcus antarcticus, Prevotella bivia, Prevotella buccae, Prevotella buccalis, Prevotella denticola, Prevotella histicola, Prevotella intermedia, Prevotella micans, Prevotella oralis, Prevotella nigrescens, Prevotella ruminicola, Prevotella stercorea, Prevotella tannerae, Prevotella timonensis, Prevotella veroralis, Ralstonia syzygii, Rhodopseudomonas palustris, Rhodospirillum rubrum, Riemerella anatipestifer, Roseburia intestinalis, Ruminococcus albis, Ruminococcus lactaris, Scardovia inopinata, Scardovia wiggsiae, Solobacterium moorei, Sphaerochaeta globus, Sphingobacterium spiritivorum, Streptobacillus moniliformis, Sutterella wadsworthensis, Treponema denticola, Tistrella mobilis, Veillonella atypica, Veillonella parvula, Weeksella virosa, Wolinella succinogenes or Zunongwangia profunda,

6. The complex according to claim 5, wherein the Cas protein is derived from S. pyogenes, S. thermophilus, S. mutans, C. jejuni, F. novicida, P. multocida or N. meningitides.

7. The complex according to claim 1, wherein the guide RNA is coupled to the Cas enzyme through an RNA linker molecule.

8. The complex according to claim 1, wherein the guide RNA is covalently coupled to the Cas protein.

9. The complex according to claim 8, wherein the covalent coupling is established by UV irradiation.

10. The complex according to claim 8, wherein the coupling is made via the backbone of the RNA molecule.

11. The complex according to claim 1, wherein the guide RNA is non-covalently complexed with the Cas protein.

12. Method for delivering a construct capable of gene editing to a eukaryotic cell, comprising the steps of:
a. providing a construct comprising a complex according to claim 1; and
b introducing said construct into said eukaryotic cell.

13. Method for gene editing a eukaryotic cell comprising providing a complex according to claim 1 to said cell.

14. Method according to claim 12, wherein said cell is part of an organism, preferably wherein the organism is selected from the group of fungi, algae, plants and animals, including humans.

15. (canceled)

16. Method for gene editing a eukaryotic cell comprising providing a complex between a Type II Cas based nuclease and a guide RNA and introducing said complex into the cell.

17. Method according to claim 16, wherein said introduction into the cell is performed by lipofection.

18. Method for gene editing a eukaryotic cell comprising providing a construct encoding a Cas based nuclease and a construct encoding a guide RNA, wherein the guide RNA is overexpressed with respect to the Type II Cas based nuclease by being expressed under control of a strong promoter.
